(19) United States
(12) Patent Application Publication
(10) Pub. No.: US 2001/0041365 A1
(43) Pub. Date: Nov. 15, 2001

(54) Methods for monitoring alcohol consumption

(76) Inventor: Michael Laposata, Boston, MA (US)

Correspondence Address: Helen C. Lockhart, Wolf, Greenfield & Sacks, P.C., Federal Reserve Plaza, 600 Atlantic Avenue, Boston, MA, 02210-2211, US

(21) Appl. No.: 09/758,099

(22) Filed: Jan. 10, 2001

Related U.S. Application Data
(60) Provisional application No. 60/175,331, filed on Jan. 10, 2000.

(51) Int. Cl. G01N 33/50

(52) U.S. Cl. 436/132; 422/61; 702/19

(57) ABSTRACT

The invention relates to methods for differentiating chronic alcoholics from acute alcoholics (i.e., binge drinkers), using levels of ethyl oleate and ethyl palmitate. Also provided are methods for determining ethanol intake, including post-mortem determination of ante-mortem ethanol intake.

### PRIORITY OF THE APPLICATION

This application claims priority under Title 35 §119(e), of United States Provisional Application No. 60/175,331, filed Jan. 10, 2000, entitled METHODS FOR MONITORING ALCOHOL CONSUMPTION, the entire contents of which are incorporated herein by reference.

### FIELD OF THE INVENTION

The invention relates to methods for monitoring alcohol consumption based on measurement of fatty acid ethyl esters.

### BACKGROUND OF THE INVENTION

Alcoholism is a disease relating to persistent alcohol abuse, alcohol dependence (i.e., addiction), and a resulting tolerance to the consumption of excessive amounts of alcohol. It is generally associated with a serious impairment in social or occupational functioning. Recent statistics show that almost 90% of the population of the United States drinks alcohol. Roughly 5-10% of these individuals will subsequently develop chronic alcohol-related symptoms and diseases. Alcoholism affects all racial, ethnic, and socioeconomic groups.

A chronic alcoholic is an individual who has consumed alcohol for an extended period of time and, as a result, is dependent upon such consumption. Chronic alcohol consumption often leads to permanent damage of organs such as the liver, pancreas and heart. As well, chronic alcoholics tend to adopt a modified, generally anti-social, behavior. Chronic alcoholics also develop a tolerance to alcohol, requiring that they drink large quantities of alcohol in order to experience alcohol-related symptoms. This tolerance also means that chronic alcoholics are able to drink larger quantities of alcohol without demonstrable short term effects in bodily functioning or behavior.

A recent study of college campuses in the U.S. revealed that anywhere from 1 to 70% of the student body actively participate in isolated incidents of occasional, excessive or acute drinking. This latter pattern of alcohol consumption has been termed “binge drinking” and those who partake in such an activity are referred to as binge drinkers. Fifty percent of male college students and thirty seven percent of female college students are classified as binge drinkers by some recent surveys. Alcohol is a factor in 40% of all academic problems, and 28% of all dropouts from American college campuses. Most importantly, it has been predicted that as many as 360,000 of the nation's 12 million undergraduates will die from alcohol-related causes while in school.

The alcohol tolerance resulting from long-term alcohol consumption affords the chronic alcoholic the ability to consume vast quantities of alcohol without serious side effects. The general population, including the binge drinkers, on the other hand, are not capable of consuming similar quantities of alcohol without serious, or even potentially life-threatening, consequences.

### SUMMARY OF THE INVENTION

Chronic alcoholics and binge drinkers display marked difference in their tolerance and response to excessive alcohol intake. The consumption of excessive levels of alcohol is potentially lethal to the binge drinker, but rarely if ever life-threatening to a chronic alcoholic. However, it is usually difficult at the time of presentation to determine into which of these two categories a subject falls, particularly since the outward manifestations of both are similar. As a result, the ability to distinguish between chronic alcoholics and binge drinkers at the time of presentation would be valuable in the diagnosis, treatment and management of these individuals.

In its broadest sense, the invention relates to methods for monitoring alcohol consumption. The invention relates to the observation that ethanol esterification products, namely fatty acid ethyl esters (FAEEs), can be used as indicia of alcohol consumption. More specifically, the invention is premised in part on the discovery that individual FAEE species are useful in differentiating a chronic alcoholic from a binge drinker.

Thus, in one aspect, the invention provides a method for distinguishing between chronic alcoholics and binge drinkers. The binge drinker who has consumed excessive amounts of alcohol in a short period of time poses a medical emergency and must be provided with immediate medical assistance. In contrast, a chronic alcoholic who has consumed a similar amount of alcohol does not require the same level of medical attention, and generally can recover from the alcoholic stupor with sleep.

In one aspect, the invention provides a method for identifying a subject who is a chronic alcoholic. The method comprises determining a concentration of ethyl oleate (O) and a concentration of ethyl palmitate (P) in a sample from a subject, and determining a P/O ratio of the concentration of ethyl palmitate (P) to the concentration of ethyl oleate (O) in the sample. According to the method, a P/O ratio less than 0.9 is indicative of a chronic alcoholic. Subjects with a P/O ratio of less than 0.9 may be recommended for detoxification therapy. Preferably, the sample is a plasma or a serum sample.

In another aspect, the invention provides a method for identifying a subject who is a chronic alcoholic by determining a concentration of ethyl oleate (O) in a sample from a subject. An ethyl oleate (O) concentration greater than 100 pmol/mL in the sample is indicative of a chronic alcoholic. Subjects with a concentration of ethyl oleate (O) greater than 100 pmol/mL in the sample may be recommended for detoxification therapy.

In yet another aspect, the invention provides a method for identifying a subject who is a chronic alcoholic by determining a concentration of total FAEE (T) in a sample and determining an O/T ratio of the concentration of ethyl oleate (O) to the concentration of total FAEE (T) in a sample. An O/T ratio greater than 0.52 is indicative of a chronic alcoholic. Subjects with an O/T ratio greater than 0.52 may be recommended for detoxification therapy.

Any of these individual methods can be used singly to identify a subject who is a chronic alcoholic. Alternatively, any combination of two or three of the above methods can also be used in making such a determination.

A number of common embodiments of the foregoing methods are envisioned. In one embodiment, the sample is a bodily fluid. The bodily fluid may be blood, urine, sputum, saliva or semen. In preferred embodiments, the sample is whole blood, plasma or serum.

In another embodiment, the sample is isolated from the subject within 4 days of the cessation of alcohol intake. In a related embodiment, the sample is isolated from the subject within 24 hours of the cessation of alcohol intake. In yet another related embodiment, the sample is isolated from the subject within 12 hours of the cessation of alcohol intake. Preferably, the subject is a human.

In yet other aspects of the invention, methods are provided for identifying a subject who is a binge drinker. In one such aspect, a method is provided for identifying a binge drinker comprising determining a concentration of ethyl oleate (O) and a concentration of ethyl palmitate (P) in a sample from a subject, and determining a P/O ratio of the concentration of ethyl palmitate (P) to the concentration of ethyl oleate (O) in the sample. A P/O ratio greater than 1.0 is indicative of a binge drinker.

In one aspect, the invention provides a method for identifying a subject who is a binge drinker based on a concentration of ethyl oleate (O) in a sample from a subject. A concentration of ethyl oleate (O) less than 100 pmol/mL in the sample is indicative of a binge drinker.

In a further aspect, a method is provided for identifying a subject who is a binge drinker comprising determining a concentration of total FAEE (T) in a sample from the subject and determining an O/T ratio of the concentration of ethyl oleate (O) to the concentration of total FAEE (T) in the sample. An O/T ratio less than 0.52 is indicative of a binge drinker.

These methods can be used individually or in combination to identify a subject who is a binge drinker. Any combination of two or three of these latter methods can be used in making such a determination.

A number of common embodiments are shared by the methods for identifying a subject who is a binge drinker. In some embodiments, the sample is a bodily fluid. The bodily fluid may be blood, including serum or plasma, urine, sputum, saliva and semen. In preferred embodiments, the sample is blood.

In embodiments relating to the identification of a subject who is a binge drinker, the sample is isolated from the subject within 2 days of the cessation of alcohol intake. In still other embodiments, the sample is isolated from the subject within 24 hours of the cessation of alcohol intake. In yet further embodiments, the sample is isolated from the subject within 12 hours of the cessation of alcohol intake.

In other embodiments, the methods further comprise providing immediate medical attention to a subject identified as a binge drinker according to the methods of the invention. In one embodiment, a subject identified as a binge drinker is placed on hemodialysis. The methods of the invention can be applied to any mammalian subject. Preferably, the subject is a human.

The invention also provides a method for determining ethanol intake comprising determining an amount of total FAEE in a liver sample of a subject, determining an amount of total FAEE in an adipose tissue sample of the subject, and adding the amount of total FAEE in the liver sample to the amount of total FAEE in the adipose tissue sample to produce a combined total FAEE amount. According to the method, a combined total FAEE amount of greater than 2000 pmol/g is indicative of ethanol intake by the subject. In a preferred embodiment, a combined total FAEE amount of greater than 10,000 pmol/g is indicative of ethanol intake by the subject. The invention also provides methods for determining ethanol intake based on the amount of total liver FAEE. In yet further aspects, the invention provides methods for determining ethanol intake based on a combination of indicia such as amount of total liver FAEE and ratio of total liver FAEE to total adipose FAEE (as described herein). In a related aspect, a combination of amount of total adipose tissue FAEE and ratio of total liver FAEE to total adipose FAEE may also be used as an indicator of ethanol intake.

The subject is preferably a human. In one embodiment, the subject is less than 2 years of age. In another embodiment, the subject is deceased. In this latter embodiment, the liver sample and the adipose tissue sample may be harvested from the subject within 5 days of death or within 3 days of death. In some embodiments, the liver sample and the adipose tissue sample are harvested from the subject within 24 hours of death.

The invention provides other methods for determining ethanol intake in a subject. In one aspect the invention provides a method for this purpose comprising determining an amount of total FAEE in a liver sample of the subject, determining an amount of total FAEE in an adipose tissue sample of the subject, and determining the ratio of the amount of total liver FAEE to the amount of total adipose FAEE. According to this method, a ratio of the amount of total liver FAEE to the amount of total adipose FAEE of at least 2 is indicative of ethanol intake by the subject. In one embodiment, the method may also take the amount of total liver FAEE into consideration, wherein an amount of total liver FAEE of at least 10,000 pmol/gram is indicative of ethanol uptake by the subject.

Another method provided by the invention for determining ethanol uptake in a subject comprises determining an amount of ethyl arachidonate in a tissue from the subject selected from the group consisting of liver tissue and adipose tissue, wherein an amount of ethyl arachidonate of at least 200 pmol/gram in the tissue is indicative of ethanol intake. In one embodiment, the method may also comprise determining the amount of total liver FAEE, with an amount of at least 10,000 pmol/gram being further indicative of ethanol uptake by the subject. In another embodiment, the method further comprises determining amounts of total liver FAEE and total adipose FAEE, with an combined amount of at least 2,000 pmol/gram and more preferably 10,000 pmol/gram or a ratio of the amount of total liver FAEE to the amount of total adipose FAEE of at least 2 is further indicative of ethanol intake.

The following embodiments relate to the methods for determining ethanol uptake by a subject. In important embodiments, the subject is predeceased and the method is used to determine pre-mortem ethanol uptake. In one embodiment, the subject is less than 2 years of age. In related embodiments, the tissue sample(s) is harvested from the subject within 5 days, within 3 days or within 24 hours of death. In preferred embodiments, the subject is a human. In some embodiments, the subject has ethanol in the blood.

In another aspect, the invention provides a method for determining ethanol intake by a subject using a single or a combination of parameters from those described herein, according to an algorithm and/or a computer system. This illustrative embodiment may be implemented as a computer program product that includes a computer-readable medium and computer-readable signals stored on the computer-readable medium, which signals define appropriate instructions. These instructions, as a result of being executed by a computer, instruct the computer to perform the Acts described herein for this illustrative embodiment.

Thus, in one aspect, the invention provides a computer program product, comprising: a computer-readable medium; and computer-readable signals stored on the computer-readable medium that define instructions that, as a result of being executed by a computer, instruct the computer to perform a process of determining ethanol intake by a subject, the process comprising steps (or acts) of determining whether combined total amount of liver and adipose FAEE is at least 2000 pmol/gram; determining whether ratio of total liver FAEE to total adipose FAEE is at least two; determining whether amount of liver or adipose ethyl arachidonate is at least 200 pmol/g; wherein i) a combined total amount of liver and adipose FAEE of at least 2000 pmol/gram, or ii) a ratio of total liver FAEE to total adipose FAEE of at least two and an amount of total liver FAEE of at least 10,000 pmol/g, or iii) an amount of ethyl arachidonate level in liver or adipose of at least 200 pmol/g, are each indicative of ethanol intake by a subject. The product is so designed as to rely on a single or a combination of these parameters in making the final determination as to whether a subject has ingested alcohol.

In one embodiment, the product further comprises the step of determining whether blood ethanol concentration is at least 10 mg/dL. In another embodiment, the product further comprises the step of determining whether urine or vitreous ethanol levels are positive for ethanol. In still another embodiment, the product further comprises the step of determining the combined total amount of liver and adipose FAEE from data entry of total amount of liver FAEE and data entry of total amount of adipose FAEE. Alternatively, the combined total amount of liver and adipose FAEE may be determined by an operator. In a related embodiment, the product further comprises the step of determining the ratio of total liver FAEE to total adipose FAEE from data entry of total amount of liver FAEE and data entry of total amount of adipose FAEE. Again, alternatively, the ratio may be determined by an operator. The product may further comprise the step of determining whether the subject was a chronic alcoholic. In another embodiment, the product may further comprise the step of determining whether the subject was a binge drinker.

In yet a further embodiment, the invention provides a method of determining ethanol intake by a subject, comprising determining whether combined total amount of liver and adipose FAEE is at least 2000 pmol/gram; determining whether ratio of total liver FAEE to total adipose FAEE is at least two; determining whether amount of liver or adipose ethyl arachidonate is at least 200 pmol/g; wherein i) a combined total amount of liver and adipose FAEE of at least 2000 pmol/gram, or ii) a ratio of total liver FAEE to total adipose FAEE of at least two and an amount of total liver FAEE of at least 10,000 pmol/g, or iii) an amount of ethyl arachidonate level in liver or adipose of at least 200 pmol/g, are each indicative of ethanol intake by a subject, and wherein the method is implemented on a computer.

In yet another aspect, the invention provides a kit for determining an amount of FAEE in a sample. The kit comprises a collection tube, a control amount of isolated FAEE, an ice pack, a thermal container, and instructions for processing the sample to determine the levels of individual or total FAEE levels. The FAEE may be selected from the group consisting of ethyl oleate, ethyl palmitate, ethyl arachidonate, and total FAEE.

### BRIEF DESCRIPTION OF THE FIGURES

FIG. 1 shows levels of serum FAEE (left panel) and blood ethanol (right panel) in men and women as a function of time after beginning alcohol consumption.

FIG. 2 compares the correlative ability of FAEE concentration (top panels) and FAEE/TG ratio (bottom panels) to blood ethanol levels in men (left panels) and women (right panels). Strength of correlation is indicated by r values. TG refers to triglycerides.

FIG. 3 shows the effect of temperature and storage time on FAEE content in a biological sample.

FIG. 4 shows total FAEE levels and blood ethanol levels as a function of time for 6 individuals who consumed a given amount of alcohol over a 2 minute time period.

FIG. 5 shows total FAEE levels and blood ethanol levels as a function of time for 7 individuals who consumed a given amount of alcohol over a 90 minute time period.

FIG. 6 shows the proportion of ethyl palmitate, ethyl stearate and ethyl oleate in chronic and binge drinkers as a percentage of total FAEE.

FIG. 7 shows the proportion of ethyl oleate relative to total FAEE in serum in chronic and binge drinkers at or near peak ethanol levels, and at 24 hours after peak ethanol levels.

FIG. 8 shows the absolute concentration of serum ethyl oleate in chronic and binge drinkers at or near peak ethanol levels, and at 24 hours after peak ethanol levels.

FIG. 9 shows the serum P/O (i.e., ethyl palmitate to ethyl oleate) ratio in chronic and binge drinkers at or near peak ethanol levels, and at 24 hours after peak ethanol levels.

FIG. 10 shows the difference in the proportion of total FAEE which is ethyl stearate and proportion of total FAEE which is ethyl oleate in chronic and binge drinkers 24 hours after ethanol consumption.

FIG. 11 shows the proportion of total FAEE which is ethyl palmitate in chronic and binge drinkers at or near peak ethanol levels, and at 24 hours after peak ethanol levels.

FIG. 12 shows the proportion of total FAEE which is ethyl stearate in chronic and binge drinkers at or near peak ethanol levels, and at 24 hours after peak ethanol levels.

FIG. 13 shows the total FAEE content in liver (left panel), in adipose tissue (center panel) and in liver and adipose tissue combined (right panel) from deceased rats administered alcohol ante-mortem.

FIG. 14 shows the total FAEE content in liver (left panel), in adipose tissue (center panel) and in liver and adipose tissue combined (right panel) from deceased humans with detectable (+ETOH) and undetectable (−ETOH) ethanol in their blood prior to death.

FIG. 15 shows the correlation between blood ethanol concentration and amount of total liver FAEE in human subjects with detectable blood ethanol at the time of autopsy (n=15).

FIG. 16 shows the correlation between blood ethanol concentration and total adipose FAEE in human subjects with detectable blood ethanol at the time of autopsy (n=14).

FIG. 17 shows the correlation between blood ethanol concentration and ethyl oleate levels in livers from human subjects with detectable blood ethanol at the time of autopsy (n=15).

FIG. 18 shows a comparison between FAEE levels in liver from human subjects with detectable blood ethanol at the time of autopsy (n=15), chronic alcoholics (n=7) and social drinkers (n=9).

FIG. 19 shows a comparison between FAEE levels in adipose tissue from human subjects with detectable blood ethanol at the time of autopsy (n=14), chronic alcoholics (n=7) and social drinkers (n=9).

FIG. 20 shows the presence of various FAEE as a percentage of total FAEE in liver tissue from human subjects with detectable blood ethanol at the time of autopsy (n=15), chronic alcoholics (n=7) and social drinkers (n=9).

FIG. 21 shows the presence of various FAEE as a percentage of total FAEE in adipose tissue from human subjects with detectable blood ethanol at the time of autopsy (n=14), chronic alcoholics (n=7) and social drinkers (n=9).

FIG. 22 shows the level of ethyl arachidonate in liver tissue from human subjects with detectable blood ethanol at the time of autopsy (n=15), chronic alcoholics (n=7) and social drinkers (n=9).

FIG. 23 shows the level of ethyl arachidonate in adipose tissue from human subjects with detectable blood ethanol at the time of autopsy (n=14), chronic alcoholics (n=7) and social drinkers (n=9).

FIG. 24 represents an algorithm or decision making process for post-mortem determination of pre-mortem ethanol intake by a subject. The approach can be similarly applied to determination of ethanol intake by a live subject.

Asterisks in the figures correspond to statistically significant differences between the experimental groups at the p values indicated.

### DETAILED DESCRIPTION OF THE INVENTION

Alcohol intake has been measured in a variety of ways, including behavioral indicia and biological marker assays. As used herein, alcohol, ethanol and ethyl alcohol, are used interchangeably. Biological markers reportedly useful in detecting alcohol and estimating the amount of alcohol consumed include ethanol content of bodily fluids and/or breath, content of carbohydrate deficient transferrin, enzymes, fatty acid ethyl ester level, and liver function tests. An example of a standard measure of the amount of alcohol a person has consumed is blood alcohol content. Blood alcohol content is the amount of alcohol in the bloodstream and is commonly expressed in milligrams of alcohol per 100 milliliters of blood, or milligrams percent. A blood alcohol content of0.10 indicates that 1/1000 of your blood volume is alcohol. Alcohol is commonly consumed in the form of beer, wine or spirits such as whisky or vodka. Alcoholic drinks contain differing proportions of alcohol. For example, beer generally is 5% alcohol by volume, wine is 11% alcohol by volume, sherry is 20% alcohol by volume and whisky is 40% alcohol by volume. Smaller volumes of high alcohol content beverages need to be consumed to experience the effects of alcohol. As a result, the same amount of alcohol is present in 355 mL of beer, 178 mL of wine, 90 m of sherry and 45 mL of whisky.

The most common response to alcohol intake is acute alcohol intoxication, otherwise known as drunkenness. Symptoms associated with drunkenness include a flushed face, a sense of euphoria, talkativeness and increased social confidence. These symptoms are induced in occasional social drinkers when blood alcohol levels range from 30-50 mg/dL. In contrast, chronic alcoholics require elevated levels of alcohol to experience identical symptoms, usually in the range of 150-200 mg/dL. Increased levels of blood alcohol (i.e., 50-150, for occasional social drinkers, and 200-350 for chronic alcoholics) tend to affect thinking and coordination, increase irritability, and reduce self-control leading to sometimes offensive talk and behavior. Even more severe symptoms are manifest at higher blood alcohol levels, including unsteady gait, slurred speech, random and extreme displays of emotion and confusion. Such characteristics can be present in occasional drinkers with 150-250 mg/dL blood alcohol levels and in chronic alcoholics with 350-500 mg/dL blood alcohol levels. When blood alcohol levels reach 250-400 mg/dL in occasional drinkers and 500-700 in chronic drinkers, extreme confusion and disorientation, inability to stand, a diminished capacity to respond to questions, and sleepiness are commonly experienced. These symptoms are reflective of the onset of a comatose state into which the subject is falling. A comatose state is a state of unconsciousness from which the subject cannot be aroused. Blood alcohol levels in the 400-500 mg/dL range for occasional drinkers impose a serious risk of death due to breathing arrest. Chronic alcoholics face such a risk only when blood alcohol level reaches in excess of 700 mg/dL, and even then these drinkers may survive.

Blood ethanol level however is not a completely robust measure of alcohol intake. For example, depending on the rate of alcohol consumption and/or the time between the cessation of alcohol intake and measurement of blood alcohol levels, the blood alcohol content may not accurately reflect the amount of alcohol the subject has ingested. Blood ethanol levels do not persist beyond 4-6 hours and thus are useful only as short term indicators of alcohol consumption. Furthermore, blood alcohol levels usually measure the level of alcohol in the blood, which provides no information about whether a subject is a chronic alcoholic or a binge drinker.

The invention, in one aspect, relates to methods for distinguishing between chronic alcoholics and binge drinkers at the time of presentation. The time at which a chronic alcoholic or a binge drinker is first monitored by medical personnel may be soon after peak alcohol levels or may be sometime thereafter. Depending on the length of time between peak alcohol level and the time of observation, mere measurement of blood alcohol levels as discussed above may not accurately reflect prior alcohol intake. Moreover, blood alcohol level cannot distinguish a chronic alcoholic from a binge drinker. Because the effect of excessive alcohol intake and the subsequent necessary course of treatment will be markedly different for these two groups of subjects, it is desirable to identify each subject accordingly. In fact, in some instances, the ability to differentiate between a chronic alcoholic and a binge drinker may be life-saving.

Thus, the invention is premised in part on the discovery that chronic alcoholics and binge drinkers can be distinguished based on content of ethanol metabolites. Ethanol can be metabolized in a mammalian system by oxidative and non-oxidative pathways. Oxidative metabolism of ethanol to acetaldehyde is performed by enzymes such as alcohol dehydrogenase, the microsomal ethanol oxidizing system of enzymes, and catalase. Acetaldehyde is then converted to acetate through the action of aldehyde dehydrogenase. Non-oxidative metabolism of ethanol involves esterification of ethanol and a fatty acid. Fatty acids are straight chain hydrocarbons, generally ranging from 10-24 carbons in length with a carboxyl terminus and a methyl terminus and having the formula CH₃(CH₂)ₙCOOH. Oleic acid is a 18:1 fatty acid meaning that it has an 18 carbon chain with one double bond. Palmitic acid, the major unsaturated fatty acid consumed in the American diet, is a 16:0 fatty acid meaning that it has a 16 carbon chain with no double bonds.

One aspect of the invention relates to methods for identifying a chronic alcoholic based on the levels and proportions of individual fatty acid ethyl ester (FAEE) species. Fatty acid ethyl esters are the esterification products of ethanol and fatty acids formed during non-oxidative metabolism of ethanol. FAEEs include but are not limited to ethyl oleate (E18:1), ethyl palmitate (E16:0), ethyl stearate (E18:0), ethyl arachidonate (E20:4), and ethyl linoleate (E18:2). These latter FAEE are the predominant FAEE species in the blood after ethanol intake, representing greater than 95% of total FAEE in the blood. (Dan, L. and M. Laposata, Alcohol Clin. Exp. Res., 21:286-292, 1997; Doyle, K. M. et al., JAMA, 276:1152-1156, 1996) Ethyl oleate is a fatty acid ethyl ester formed from the esterification of the fatty acid oleic acid and ethanol. Ethyl palmitate is a fatty acid ethyl ester formed from the esterification of the fatty acid palmitic acid and ethanol. Total FAEE, as used herein, refers to the total of all FAEE species including but not limited to ethyl oleate, ethyl palmitate, ethyl stearate, ethyl linoleate and ethyl arachidonate. FAEE can be synthesized by FAEE synthase. FAEE synthase is found in highest levels in the pancreas and liver, both organs which are known to suffer the greatest damage as a result of ethanol abuse. Other enzymes known to synthesize FAEE include carboxylesterase from adipose tissue and pancreas, lipoprotein lipase, pancreatic triglyceride lipase, and cholesterol esterase.

FAEE appear in the blood very soon after alcohol intake. Thus, like blood alcohol level, serum FAEE level is a useful indicator of alcohol intake in the early time points after alcohol intake (i.e., 6 hours). However, unlike blood alcohol level, serum FAEE persists beyond 6 hours after alcohol intake and thus is a useful marker for determining alcohol intake in the long-term. Moreover, FAEEs have been shown to be a far more sensitive marker for alcohol intake, since at low levels of alcohol consumption resulting in baseline (and thus, negative) blood ethanol readings, serum FAEE readings are above baseline. A positive blood ethanol level invariably correlates with a positive FAEE level. The converse (i.e., that a positive FAEE measurement will be reflected by a positive blood alcohol measurement) may not be true. Total or individual FAEE content is determined in a bodily sample according to methods known in the art and described in detail herein. Irrespective of their utility in determining prior alcohol intake, FAEE have been reported to mediate ethanol induced organ damage particularly to organs which have high levels of FAEE synthase, such as the liver and pancreas.

Chronic alcoholics are individuals with a history of long-term, habitual and compulsive consumption of excessive levels of alcohol. According to the Diagnostic and Statistical Manual of Mental Disorders, the presence of three or more of the following criteria is indicative of an alcoholic: 1) increased tolerance, 2) withdrawal, 3) drinking more often and drinking larger quantities, 4) wanting but being unable to quit, 5) spending much time getting alcohol, 6) abandonment of other activities in favor of drinking, and 7) continuing to drink even though experiencing adverse side effects (DSM IV, page 81, 1994). Chronic alcoholics generally experience withdrawal symptoms once they stop drinking. Long-term effects of chronic alcoholism include alcohol related disorders such as liver diseases, heart diseases and hypertension. The organs most commonly damaged by ethanol abuse are the pancreas and the liver.

Typically, chronic alcoholics have an increased tolerance for alcohol based on this past history. The basis of this alcoholic tolerance is multi-faceted and can include metabolic or pharmacokinetic tolerance, cellular or pharmacodynamic tolerance and behavioral tolerance. Metabolic tolerance, which relates to the metabolism of alcohol by the liver, can be induced with as little as 1-2 weeks of daily drinking. As a result of the increase in the metabolism of alcohol by the liver, the alcoholic must consume more alcohol in order to maintain the blood alcohol levels necessary to experience the effects of the alcohol. Once alcohol consumption ceases, however, this type of tolerance dissipates just as quickly as it originally arose. Physical dependence invariably results from chronic alcohol consumption and relates to changes in cell membrane permeability which underlie cellular tolerance. Behavior tolerance refers to the ability of subjects to adapt to the increased levels of alcohol leading to an improved motor control performance when under the influence of alcohol as compared to when sober. Neurons are particularly sensitive to these alcohol changes. Reversal of the alcohol dependence may take weeks or longer. As used herein, chronic alcoholism refers to habitual, compulsive, long-term and excessive consumption of alcohol by a subject. In some instances, chronic alcoholism may be associated with the experience of withdrawal symptoms once alcohol consumption has stopped.

One parameter for the identification of a chronic alcoholic is the serum or plasma concentration of ethyl oleate in a sample from the subject. The concentration of ethyl oleate can be determined by methods known in the art. Such methods are described herein, and include gas chromatography alone or in combination with mass spectrometry. Ethyl oleate concentration is represented by the amount of ethyl oleate (pmol) in a one mL volume sample of bodily fluid. Thus the final measurement is provided in units of pmol/mL. The denominator value in this measurement corresponds to the volume of the sample of bodily fluid which is tested. A concentration of ethyl oleate in excess of 100 pmol/mL in a sample from a subject is indicative of a chronic alcoholic. As an illustrative example, an individual with more than 100 pmol of ethyl oleate in a one mL sample of their blood is considered a chronic alcoholic.

A second parameter useful for identifying chronic alcoholics is the proportion of total fatty acid ethyl esters represented by ethyl oleate. For this determination, both total FAEE (T) and ethyl oleate (O) concentrations are determined for a given volume of an identical bodily fluid sample. Total FAEE concentration is measured using the same approach as that used to measure the concentration of individual FAEEs, and the sum of the concentrations of individual FAEEs is used. The proportion of total FAEE which is ethyl oleate can also be referred to as the ratio of the concentration of ethyl oleate to the concentration of total FAEE. This ratio is referred to herein as the O/T ratio. An O/T ratio greater than 0.52 is indicative of a chronic alcoholic. That is to say, when 52% or more of the total FAEE content in a sample is ethyl oleate, the subject from whom the sample was harvested is considered a chronic alcoholic.

The third parameter for identifying a chronic alcoholic is the ratio of the concentration of ethyl palmitate (P) to the concentration of ethyl oleate (O) in a sample from a subject. This ratio, which is referred to herein as the P/O ratio, is determined by measuring the concentrations of ethyl palmitate and ethyl oleate in a sample from a subject, again using a method identical to that described for ethyl oleate alone or for total FAEE. The P/O ratio is determined by dividing the concentration of ethyl palmitate into the concentration of ethyl oleate present in a given sample size. A P/O ratio of less than 0.9 is indicative of a chronic alcoholic.

Chronic alcoholics can be identified according to the invention using any one of three different parameters presented herein. The invention similarly provides for the identification of a chronic alcoholic using any two or all three of these parameters in combination.

If the subject is identified as a chronic alcoholic, then detoxification therapy may be recommended. Subjects identified as chronic alcoholics upon presentation to medical staff should be provided detoxification therapy. Detoxification therapy is the medical help necessary to overcome the withdrawal symptoms that will invariably be experienced by a chronic alcoholic upon the cessation of alcohol consumption. Detoxification can sometimes include the administration of drugs which simulate the alcohol effect but without the lasting long-term side effects of alcohol. Detoxification should be followed by long-term therapy which can include psychological, social and physical therapies. An example of a physical therapy is the administration of disulfiram, a drug which induces nausea in a person who ingests alcohol.

The methods of the invention are also useful for identifying subjects who are binge drinkers. Binge drinkers are those subjects who partake in isolated incidents of excessive alcohol consumption. More specifically, a binge drinker, as used herein, refers to an individual who consumes several alcoholic drinks in a short period of time (e.g., within a 4 hour time-span), but who is not a chronic alcoholic. Since binge drinkers do not have a history of persistent alcohol abuse, they have yet to develop a tolerance for high alcohol intake. Tolerance for excessive alcohol consumption is therefore a hallmark of a chronic alcoholic. One measure of a binge drinker is a male subject who consumes 5 or more alcoholic beverages in a row in a short period of time. Another measure of a binge drinker is a female subject who consumes 4 or more alcoholic drinks in a row in a short period of time. The amount of alcohol consumed is set at 5 or more drinks for a male and 4 or more drinks for a female since this is the amount of alcohol which causes an individual to experience 10 times greater negative consequences relative to when they are sober. Such negative consequences may impact upon health, relationships, legal problems, behavior problems, accidents, injuries, assaults and, in the most extreme situations, death. The amount of alcohol required for a woman to binge drink is lower since women begin to experience such risks upon lesser consumption of alcohol. Consumption of the same amount of alcohol in a shorter period of time will probably result in more severe alcohol-related symptoms. According to these definitions, the average alcoholic beverage can generally be defined as a half-ounce of alcohol. This amount of alcohol is present in a 12-ounce can of beer, a 4-ounce glass of table wine, or a 1 -ounce shot of 100-proof distilled spirits such as whiskey or vodka. This amount of alcohol is generally the amount that the average, non-chronic alcoholic, person can metabolize in one hour. Binge drinking is common in subjects in their early teens to late twenties. For some individuals, several binge drinking episodes (i.e., if female, the consumption of 4 or more drinks, or if male, 5 or more alcoholic beverages, in a short period of time) may occur in a two week time span.

Binge drinking is associated with several alcohol-related behaviors including anxiety, arguments, fights and depression. Not surprisingly, persons engaged in binge drinking do not consider themselves to have a problem with alcohol. This latter observation underscores the importance of effective identification of binge drinkers for the purpose of short- and long-term treatment. Such incidences of binge drinking are frequently associated with blackouts, impaired behavior (e.g., argumentative natures), severe nausea, vomiting, dizziness, impaired mental capabilities and hangover. Secondary and long-term effects of binge drinking include effects on athletic performance, poor grades, sexual assault, fights, accidents, drunk driving, and a variety of health risks.

The most important concern relating to binge drinking is alcohol poisoning. Alcohol poisoning is a condition in which a subject has ingested alcohol at an excessive rate or in excessive quantities and as a result gets severely ill. Symptoms relating to alcohol poisoning include non-responsiveness to either verbal or physical contact, inability to stand, inability to be aroused or awoken, slow, labored or abnormal breathing, purplish discoloration of the skin, “clammy” skin, increased pulse rate, irregular heart rhythm and lowered blood pressure. The risk of a binge drinker choking on his/her own vomit during this time is also increased.

Thus the invention in part aims to identify subjects who are binge drinkers. The methods for identifying a subject who is a binge drinker are similar to those used to identify a chronic alcoholic as described herein. One method for identifying a subject who is a binge drinker involves determining a concentration of ethyl palmitate in a sample from the subject. A concentration of ethyl oleate which is less than 100 pmol/mL may be indicative of a binge drinker. Concentrations of ethyl oleate less than 2, 5, 10, 20, 30, 40, 50, 75, 90 and 100 pmol/mL are indicative of a binge drinker. Preferably, a concentration of ethyl oleate less than 100 pmol/mL is indicative of a binge drinker.

Another method for identifying a binge drinker involves measuring concentrations of ethyl palmitate (P) and ethyl oleate (O) in a sample and determining the ratio of the concentration of ethyl palmitate (P) to the concentration of ethyl oleate (O) (i.e., the P/O ratio). P/O ratios greater than 8, greater than 5, greater than 2, and greater than 1 can each be used as indicators of a binge drinker. Preferably, a binge drinker is indicated by a P/O ratio greater than 1.0.

In a third method for identifying a subject who is a binge drinker, a concentration of ethyl oleate (O) and a concentration of total FAEE (T) are determined in a sample from a subject. The ratio of the concentration of ethyl oleate to the concentration of total FAEE (i.e., the O/T ratio) in the sample is used to determine if the subject is a binge drinker. O/T ratios less than 0.52, less than 0.50, less than 0.40, less than 0.30, less than 0.20, less than 0.10, and less than 0.05 may each be indicative of a binge drinker. Preferably, an O/T ratio less than 0.52 is indicative of a binge drinker.

Any of these three methods may be used individually or in combination to identify a subject who is a binge drinker. If, using the methods of the invention, the subject is identified as a binge drinker, then the subject is provided immediate medical attention. Such medical attention may be aimed at preventing further absorption from the gastrointestinal tract in the subject or enhancing elimination of alcohol from the subject. Prevention of further absorption can be effected by administering syrup of ipecac to induce vomiting, gastric lavage, administration of activated charcoal by stomach tube or mouth, catharsis, and dilution. Methods for enhancing elimination of alcohol include administration of multiple dose activated charcoal, forced diuresis, peritoneal dialysis, hemodialysis, hemoperfusion, hemofiltration, plasmapheresis and exchange transfusion. A preferred element of immediate medical assistance provided to the binge drinker is hemodialysis. Hemodialysis refers to the dialysis of blood for the purpose of removing toxins by artificial filtration. In some instances, the blood is treated by circulating it through an artificial kidney machine. It is recommended that in some cases, the binge drinker is admitted to an intensive care unit and monitored.

The afore-mentioned methods of the invention share a number of common embodiments. Preferably, the subject is a human. The sample from the subject may be a bodily fluid including but not limited to blood either whole or in serum or plasma form, lymph, saliva, urine, sputum, semen and the like. The preferred body fluid is blood either in whole, serum or plasma form.

The sample can be harvested from a subject at various times. If the subject is to be monitored for chronic alcoholism or for a binge drinking event, then the sample can be harvested from the subject at or after peak ethanol times. In the case of human subjects, subjects will present in a hospital or a clinic at or near peak ethanol levels if intoxication is their primary complaint. If accompanied with another person, it may be possible to determine the time at which drinking ceased. If not accompanied by another, however, the patient is considered to be at or near peak ethanol levels if intoxication is their primary complaint. Peak ethanol times as used herein refer to the time at which the subject has maximum levels of ethanol in the blood. Blood ethanol levels can be deduced through a number of methods known in the art. An example of a method for blood alcohol measurement is described in more detail below. Peak ethanol levels generally occur 20-60 minutes after drinking has ceased, and alcohol presence in the blood persists for about 4-6 hours. Peak FAEE levels occur within 40-60 minutes after drinking has ceased, and FAEE presence in the blood persists usually for more than 6 hours. In other embodiments, the sample may be harvested from the subject within 4 days, within 3 days or within 2 days after the time of peak ethanol levels. Preferably, the sample is harvested from the subject within 24 hours, or within 12 hours after peak ethanol times. In preferred embodiments, the sample is harvested within 24 hours after peak ethanol times. If the subject is suspected of binge drinking, it is preferred that the sample be harvested at earlier time points such as within 48, within 36, within 24 or within 12 hours of peak ethanol times so that medical assistance can be provided promptly.

The concentrations of ethyl oleate (O), ethyl palmitate (P), and total FAEE (T) can be readily determined using methods well known in the art. Described herein is an exemplary method which may be used to determine levels of total or individual FAEE species. The methods of the invention however are not so limited to the use of only this method; rather it is intended that any method known in the art for detecting and measuring FAEE levels may be used.

Thus, as an example, gas chromatography either alone or in combination with mass spectrometry and/or an initial step of solid phase extraction can be used to determine total and/or individual fatty acid ethyl ester concentrations. (Bernhardt, T. G., et al., J. Chromatography B, 675:189-196, 1996) Using this technique, recovery of 70+/−3% of initial amounts of ethyl oleate have been achieved. Typically a sample such as, for example, a blood sample is harvested from a subject. If the sample has been previously harvested and frozen, it is thawed out and a 1 mL aliquot is removed for analysis. The aliquot may be centrifuged for 10 minutes at 1250 g in order to isolate serum, however no statistically significant difference in FAEE concentration has been observed between serum and plasma. In order to determine and quantitate FAEE content, an internal standard of 1 nmol of ethyl heptadecanoate (ethyl 17:0) is added to each sample. Samples are extracted with acetone hexane (2:8, by volume) and dried under nitrogen vapors to a volume of approximately 300 μL. FAEEs are isolated by solid phase extraction using Bond Elut-LRC aminopropyl columns. Columns are pre-washed with dichloromethane followed by hexane; the sample is then applied to the column and eluted by successive washes of hexane and dichloromethane. Combined eluates are concentrated, and FAEEs are then quantified by GC-MS using a Hewlett Packard 5890 Series II gas chromatograph equipped with a Supelcowax SP-2330 capillary column coupled to an HP-5971 mass spectrometer. The injector and detector are maintained at 260° C. and 280° C., respectively. The oven program is initially maintained at 130° C. for 2 minutes then ramped to 160° C. at 5° C./min, ramped again at 2° C./min to 180° C., held for 7 min, finally, ramped to 230° C. at 15° C/min and maintained for 2 min. Carrier gas flow rate is maintained at a constant 0.8 mL/min throughout. Single-ion monitoring is performed, quantifying appropriate base ions for individual FAEE species [i.e., ions m/z 67, 88, and 101 for ethyl palmitate (E16:0), ethyl heptadecanoate (E17:0), ethyl stearate (E18:0), ethyl oleate (E18:1), and ethyl linoleate (E18.2); and ions m/z 79 and 91 for ethyl arachidonate (E20:4), ethyl eicosapentaenoate (E20:5), and ethyl docosahexaenoate (E22:6)]. FAEEs are quantitated by interpolation of the slope generated from individually prepared calibration curves comparing areas of varying concentrations of E16:0-E22:6 to fixed concentrations of the internal standard (E17:0). Mass relationships are obtained for each FAEE using its individual calibration curve. Total FAEE mass is determined by the addition of masses of individual FAEEs (E16:0-E22:6).

Thin layer chromatography (TLC) can also be used to separate and quantitate individual FAEE species and is preferably if the samples are radiolabeled. Lipids are extracted using a modified Folch method. (Cohen et al., J. Clin. Invest., 50:762-772, 1971) FAEEs are subsequently isolated from the organic phase using thin-layer chromatography (TLC). The recommended solvent is a 75:5:1 mixture of petroleum ether, diethyl ether and acetic acid. The samples are run on silica gel 60 plates. The FAEEs so isolated are scraped from the TLC plates under nitrogen and then eluted from the silica gel with 2.5 mL of acetone. Eluted FAEEs are concentrated by drying under nitrogen. A 1-2 μL sample is then loaded onto a Perkin-Elmer 8500 GC with a WCOT Supelcowax capillary column. FAEEs are separated and quantitated using a temperature program from 150° C.-250° C. increasing at 10° C. per minute. FAEEs can then be identified by comparison to known FAEE standard which can be purchased from Nu-Check Prep, Elysian, Minn. Several FAEE species, including E16:0, E16:1 n-7, E18:0, E18:1 n-9, E18:2 n-6, and E20:4 n-6 can be resolved from their corresponding fatty acid methyl esters using this approach.

In a parallel approach to determining FAEE content, lipids can be extracted and separated using TLC as described above, following which individual FAEEs can be identified by gas chromatography-mass spectrometry (GC-MS). In some instances in which radiolabeled FAEEs are analyzed, TLC alone is preferable without the subsequent step of GC-MS analysis. GC-MS analysis is carried out on a Hewlett-Packard 5890 gas chromatograph coupled to a Hewlett-Packard 5970 mass spectrometer with a WCOT Supelcowax capillary column. The injector is maintained at 260° C., the MS detector at and maintained at 250° C. for 6 minutes. Total ion chromatograms can be generated using an ionization energy of 70 eV.

Total FAEE levels can be calculated by determining the concentration of each individual FAEE species and then adding these concentrations together to arrive at a total FAEE concentration.

Plasma ethanol concentrations can be determined by gas chromatography (GC). A blood sample is first centrifuged for 10 minutes at 1250 g in order to isolate serum. The resulting serum fraction can then be stored at −80° C. for up to 3 months. Briefly, a plasma sample is mixed with an internal standard, 1-propanol, and a 1-μL sample is injected into a Hewlett Packard 5890 GC equipped with a 5% Carbowax 20 M 60/80 Carbopack B column. The oven program is isothermal at 100° C., and the ethanol peak is identified by comparison with a known standard.

In another aspect, the invention provides a method for determining whether a subject has ingested alcohol. In preferred embodiments, the subject is one who has consumed alcohol ante-mortem (i.e., pre-mortem) with the determination being made post-mortem. In this latter regard, the invention provides a method for determining whether the subject had consumed alcohol prior to death and, in some instances, whether the death may have been caused by alcohol poisoning. The method specifically involves measuring the sum or combined total FAEE content of the liver and adipose tissue taken from the subject. Methods for determining ethanol intake that rely on analysis of tissues other than blood are useful because commonly blood ethanol levels can simply be a manifestation of bacterial metabolism in the subject rather than true ethanol intake prior to death. In addition, depending upon the treatment of the cadaver or the time between death and autopsy, there may be no blood sample available for analysis. Thus, the invention overcomes these limitations by providing methods use liver and adipose tissue, instead of blood.

As an example, samples of similar mass (e.g., 5 grams) are harvested from liver and adipose tissue and processed separately. The final measure is a sum total of the amount of combined FAEE in a fixed mass of liver and adipose tissue combined. The adipose tissue may be harvested from any region of adipose tissue in the body, including the panniculus, the gluteal and abdominal areas, and the omental and mesenteric adipose tissues. Harvest from either the liver or the adipose tissue may be performed by needle biopsy (i.e., needle aspiration). However, if the area is to be cleaned prior to injection, it is recommended that alcohol not be used since this may impact upon the results of the assay. Samples may be stored at −70° C. until processing. Adipose tissue is generally extracted in acetone followed by thin layer chromatography and gas liquid chromatography to identify individual species as described above. Liver samples may be first disrupted either mechanically (e.g., through a micron screen) or enzymatically (e.g., by treatment with a protease such as collagen or trypsin) prior to acetone extraction. Mechanical disruption is the preferred method. Total FAEE content of the liver and adipose tissue samples may be determined using the gas chromatography method described herein. The combined FAEE amount in the liver and the adipose tissue is the sum of the amount of FAEE in a sample of liver tissue and the amount of FAEE in the sample of adipose tissue. The FAEE detected and measured in the liver and adipose tissue may be individual FAEE species, but preferably it is the total FAEE amount. Thus the term combined total FAEE refers to the sum of the amount of total FAEE in the liver and the amount of total FAEE in adipose tissue. Alcohol intake ante-mortem in a human subject may be indicated by a combined total FAEE amount of greater than 1000 pmol/g, greater than 2000 pmol/g, and greater than 5000 pmol/g. Preferably, a combined total FAEE amount of greater than 2000 pmol/g, and even more preferably greater than 10,000 pmol/g, is used to indicate ante-mortem ethanol intake by a subject.

As discussed in Example 9, the invention provides markers of ethanol uptake in addition to total combined liver and adipose tissue FAEE level, and these include the ratio of the amount of total liver FAEE to the amount of total adipose FAEE, amount of total liver FAEE, and the presence of one particular form of FAEE, namely ethyl arachidonate, in either or both liver and adipose tissue samples. With respect to the ratio of total liver FAEE to total adipose FAEE, a value of 2 or more is indicative of a subject having ingested ethanol pre-mortem. With respect to the amount of total liver FAEE, a value of at least 10,000 pmol/g is also indicative of ethanol intake. In some embodiments, it may be desired to use the latter two measurements in combination when determining ethanol intake. With respect to ethyl arachidonate levels in liver or adipose tissue, a level of at least 200 pmol/g is indicative of ethanol intake. The parameters can be measured and determined using the techniques provided herein, and can be used individually or in combination with each other to determine ethanol intake by a subject. The invention also embraces the use of FAEE levels in adipose tissue as a marker of ethanol intake. If a determination of ethanol intake is being made based on a single indicia, then it may be desirable to select a higher end value so as to provide for greater discriminatory power. If a number of parameters are being used together to determine ethanol intake, then lower cut-offs of positivity may be useful. In addition, values may be dependent upon the particular species of subject being analyzed, and in some instances it may be appropriate to establish the cut-off values in control subjects. For human subjects, the values provided herein are appropriate, and no further calibration is required. Based on the teachings provided herein, establishing cut-offs values for non-human subjects is routine and well within the realm of the ordinary artisan.

According to these latter methods for determining ethanol intake by a subject, a subject includes non-human primate, dog, cat, bird, cow, pig, sheep, horse, goat and rodent. Preferably, the subject is a human. The sample from the subject may be a bodily fluid including but not limited to blood either whole or in serum or plasma form, lymph, saliva, urine, sputum, semen and the like or it may be a tissue such as liver, pancreas, adipose tissue, heart, lung, brain and kidney. In preferred embodiments, the samples are harvested from liver and adipose tissue.

The tissue samples can be harvested at various times following death. For example, the samples can be harvested within 7 days, within 6 days, within 5 days, within 4 days, within 3 days, within 2 days or within 24 hours of death. In important embodiments, the sample is harvested within 24 hours of death. In other important embodiments, the sample is harvested within 12 hours of death. Generally, if either the harvest or the analysis of bodily fluid or tissue is delayed, or if there is concern about bacterial contribution to the ethanol measurement, it may not be possible to make such a determination based on blood ethanol levels. Moreover, if the blood has coagulated, then the ability to determine alcohol intake using tissues such as liver and adipose tissue is beneficial. Harvest of tissues may be made at autopsy. The determination may also be made from a living subject. In this latter instance, the sample may be harvested by biopsy.

This latter method is particularly suited to determining cause of death or responsibility for any action taken by the subject just prior to death. As an example, the ability to determine alcohol intake just prior to death resulting from a motor vehicle accident may be useful in determining if the subject was driving under the influence of alcohol.

In another example, the methods of the invention may be used to determine whether newborn infant had been exposed to alcohol in utero as a result of alcohol intake by the mother. Alcohol related death is often times suspected in the death of young children, especially infants. Alcohol is sometimes, inappropriately, given to infants in order to soothe them. The invention in one aspect provides a method for determining on autopsy whether a deceased infant was provided alcohol.

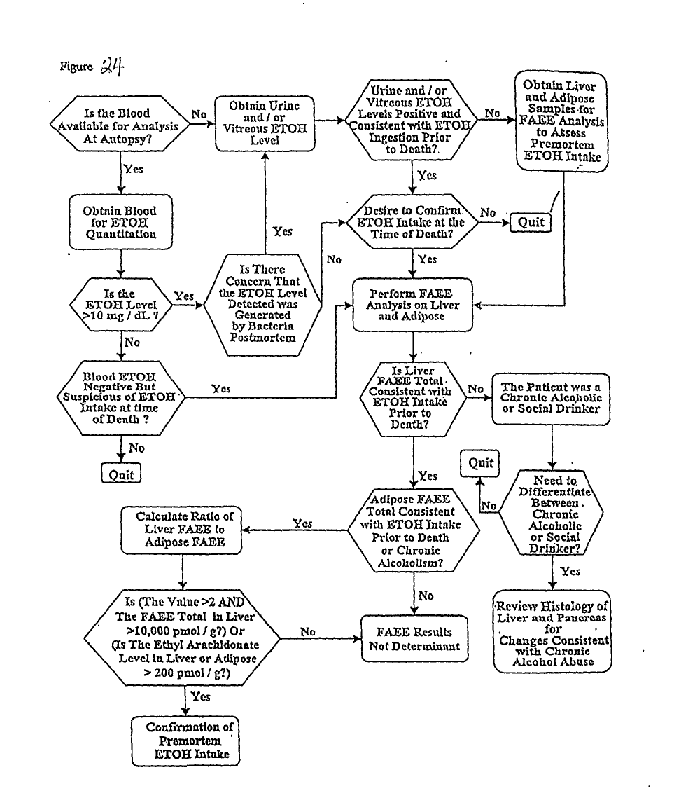

Based on the teachings provided herein, the invention further provides an algorithm for post-mortem analysis of a subject for pre-mortem uptake of ethanol. FIG. 24 illustrates one example of such an algorithm. It is to be understood that this type of algorithm can be used both for live and deceased subjects in order to determine ethanol intake provided samples are available for analysis. Moreover, it is also to be understood that the algorithm of FIG. 24 represents but one pathway for determining ethanol intake, and that equivalent determinations may be made by subtle permutations to this decision making process. For example, if neither blood, nor urine, nor vitreous fluid is available for analysis, the algorithm is still useful and the decision process simply bypassing these data points.

As shown in FIG. 24, the algorithm begins with a test for blood ethanol. If the blood ethanol is greater than 10 mg/dL in an autopsy from which a sample for blood ethanol is available, the question of whether or not the detected ethanol was generated by bacteria can be further pursued with liver and adipose FAEE levels. An amount of ≧10,000 pmol/g for liver FAEE, and a value of 2 or more for the ratio of liver FAEE/adipose FAEE provide strong evidence of pre-mortem ethanol intake. Since, as shown in Example 9, ethyl arachidonate was found only in individuals with detectable blood ethanol level at the time of autopsy, a value of ≧200 pmol/g in liver or adipose tissue also indicates pre-mortem ethanol intake. Ultimately, these three indices can be used as postmortem markers for pre-mortem ethanol intake, either in cases where there is no blood available to determine the ethanol level, or if there is a desire to confirm the blood ethanol level.

The determination of ethanol intake may be performed by a person manually, or alternatively, it may be performed by a computer process using a computer system and a computer algorithm (such as a computer program) which calls for entry of data are various points in a decision making pathway, such as that shown in FIG. 24. In this latter embodiment, the operator is required to enter data and the computer system and program provides the final determination as to whether a subject has ingested alcohol. The system is designed to allow for a determination of ethanol intake based on single or multiple parameters. Preferably, the operator enters as many parameters as are available.

Thus, the invention also provides for the implementation of an algorithm for determining ethanol intake by a subject using a typical computer system. The implementation of the algorithm is not limited to any specific computer such as that described herein, as many other different machines may be used to implement the algorithm.

Such a computer system may include a processing unit which performs a variety of functions in a manner well-known in the art in response to instructions provided from an application program. The processing unit may function according to a program known as the operating system, of which many types are known in the art. The steps of an application program typically are provided in random access memory (RAM) in machine-readable form because programs typically are stored on a non-volatile memory, such as a hard disk or floppy disk. After an application program is selected, for example, by a user, it may be loaded from the hard disk to the RAM, and the processing unit may proceed through the sequence of instructions of the application program.

The computer system also may include a user input/output (I/O) interface. The user interface typically includes a display apparatus, such as a cathode-ray-tube (CRT) display and an input device, such as a keyboard or mouse. A variety of other known input and output devices may be used, such as speech generation and recognition units, audio output devices, etc.

The computer system also may include a video and audio data I/O subsystem. Such a subsystem is well-known in the art and the algorithm for determining ethanol intake is not limited to a computer system including the specific subsystem described herein. The audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

One or more output devices may be connected to the computer system implementing the algorithm for determining ethanol intake. Example output devices include a cathode ray tube (CRT) display, liquid crystal displays (LCD) and other video output devices, printers, communication devices such as a modem, storage devices such as disk or tape, and audio output. One or more input devices may be connected to the computer system. Example input devices include a keyboard, keypad, track ball, mouse, pen and tablet, communication device, and data input devices such as audio and video capture devices and sensors. The computer system is not limited to the particular input or output devices described herein as being used in combination with the computer system.

The algorithm may be implemented on a general purpose computer system which is programmable using a computer programming language, such as “C++,” JAVA or other language, such as a scripting language or even assembly language. The computer system also may include specially programmed, special purpose hardware such as, for example, an application-specific integrated circuit (ASIC).

In a general purpose computer system, the processor is typically a commercially available processor, for example, one of the series x86, Celeron and Pentium processors, available from Intel, similar devices from AMD and Cyrix, the 680X0 series microprocessors available from Motorola, and the PowerPC microprocessor from IBM. Many other processors are available. Such a microprocessor executes a program called an operating system, of which WindowsNT, Windows95 or 98, UNIX, Linux, DOS, VMS, MacOS and OS8 are examples, which controls the execution of other computer programs and provides scheduling, debugging, input/output control, accounting, compilation, storage assignment, data management and memory management, and communication control and related services. The processor and operating system define a computer platform for which application programs in high-level programming languages are written.

A computer system that implements an algorithm for determining alcohol intake may include a memory system, which typically includes a computer readable and writeable non-volatile recording medium, of which a magnetic disk, a flash memory and tape are examples. The disk may be removable such as, for example, a floppy disk or a read/write CD, or permanent, for example, a hard drive. A disk has a number of tracks in which signals are stored, typically in binary form, i.e., a form interpreted as a sequence of one and zeros. Such signals may define an application program to be executed by the microprocessor, or information stored on the disk to be processed by the application program. Typically, in operation, the processor causes data to be read from the nonvolatile recording medium into an integrated circuit memory element, which is typically a volatile, random access memory such as a dynamic random access memory (DRAM) or static memory (SRAM). The integrated circuit memory element allows for faster access to the information by the processor than does the disk. The processor generally manipulates the data within the integrated circuit memory and then copies the data to the disk after processing is completed. A variety of mechanisms are known for managing data movement between the disk and the integrated circuit memory element, and the algorithm for determining ethanol intake is not limited thereto. The algorithm for determining ethanol intake is not limited to a particular memory system.

The algorithm for determining ethanol intake may be implemented in software or hardware or firmware, or any combination of the three. The various elements of the algorithm, either individually or in combination may be implemented as a computer program product tangibly embodied in a machine-readable storage device for execution by a computer processor. Various steps of the process implemented by the algorithm may be performed by a computer processor executing a program tangibly embodied on a computer-readable medium to perform functions by operating on input and generating output. Computer programming languages suitable for implementing such a determination of ethanol intake (as shown for example in FIG. 24) include procedural programming languages, object-oriented programming languages, and combinations of the two.

The algorithm for determining ethanol intake is not limited to a particular computer platform, particular processor, or particular programming language. Additionally, the computer system may be a multi-processor computer system or may include multiple computers connected over a computer network. The steps described in FIG. 24 may be separate modules of a computer program, or may be separate computer programs. Such modules may be operable on separate computers.

In yet another aspect, the invention embraces a kit for processing bodily fluid or tissue samples for the purpose of detecting and measuring FAEE content. The kit preferably includes an ice pack, a thermal container, a collection tube such as a vacutainer, a control amount of isolated FAEE, and instructions for processing the sample to determine FAEE content within the sample. The control amount of isolated FAEE may be introduced into the collection tube just prior to or immediately after the harvest of the bodily fluid or tissue. Alternatively, the collection tube may be manufactured to contain a known amount of total and/or individual FAEEs. In this latter embodiment, another tube containing an identical amount of total or individual FAEE as present in the collection tube may also be incorporated into the kit. In another embodiment, the control amount of FAEE may be kept separate from the harvested bodily fluid or tissue (i.e., the test sample), but still be stored and processed in the same manner as the test sample. Preferably, the control will contain a known amount of individual FAEEs. The control acts as an internal standard to determine if, and the extent to which, total and/or individual FAEEs are lost during storage or processing. For some kits, it may be preferable that the control contain known amounts, and thus a known ratio, of ethyl oleate and ethyl palmitate. Using this latter control, changes in the ratio of these individual FAEEs as a result of storage or processing can be determined. Such changes would clearly affect the interpretation of the amounts and ratios within the test sample and the ultimate determination as to whether the subject is a chronic alcoholic or a binge drinker or whether the subject has even ingested alcohol. The kit may also contain a sterile needle and syringe for the harvest of the bodily fluid or tissue. In another embodiment, the kit may contain a cleansing swab or tissue for disinfecting the surface to be perforated with the needle. It is recommended that the cleansing swab or tissue is one which does not contain alcohol in order to prevent the contamination of the sample with alcohol not endogenous to the sample. The kit is generally housed in an exterior packaging which may be, but is not limited to, a box or a bag for holding all the components of the kit including the collection tube, the ice pack, the thermal container and the instructions. Individuals skilled in the art can readily modify the packaging to suit individual needs.

### EXAMPLES

### Example 1

### Effects of Gender on Serum Fatty Acid Ethyl Ester (FAEE) Levels

Seven volunteers (four men and seven women) were given measured amounts of 100-proof vodka mixed with fruit juice in a 1:3 ratio which was weight-adjusted to increase blood ethanol levels to approximately 120-147 mg/dL. The beverage was divided in 9 equal aliquots which the subjects drank over a 90 minute period, 1 aliquot every 10 minutes. This alcohol intake represented consumption to the legal limit of intoxication. Once the drinking commenced, blood was collected from a catheter every 15 min for the first 2.5 h. Between 2.5 and 4.0 h, samples were collected every 30 min; and from 4 to 8 h, they were collected every 60 min. A final blood sample was also collected 24 h after the ethanol ingestion commenced. Blood was collected from a peripheral venous catheter into vacuum tubes without anti-coagulant (for serum FAEE) and into tubes with the anti-coagulant sodium citrate (for plasma ethanol). (Soderberg, et al., Clinical Chemistry, 45(12):2183-2190, 1999; Doyle, K. M. et al., JAMA, 276:1152-1156, 1996)

All samples were extracted with acetone/hexane (2/8, v/v) and dried under nitrogen vapors to a volume of approximately 300 μL. FAEEs were isolated by solid phase extraction using Bond Elut-LRC aminopropyl columns. FAEEs were then quantitated by gas chromatography-mass spectrometry (GC-MS) using a Hewlett-Packard 5890 Series II gas chromatograph equipped with a Supelcowax SP-2330 capillary column coupled to an HP-5971 mass spectrometer.

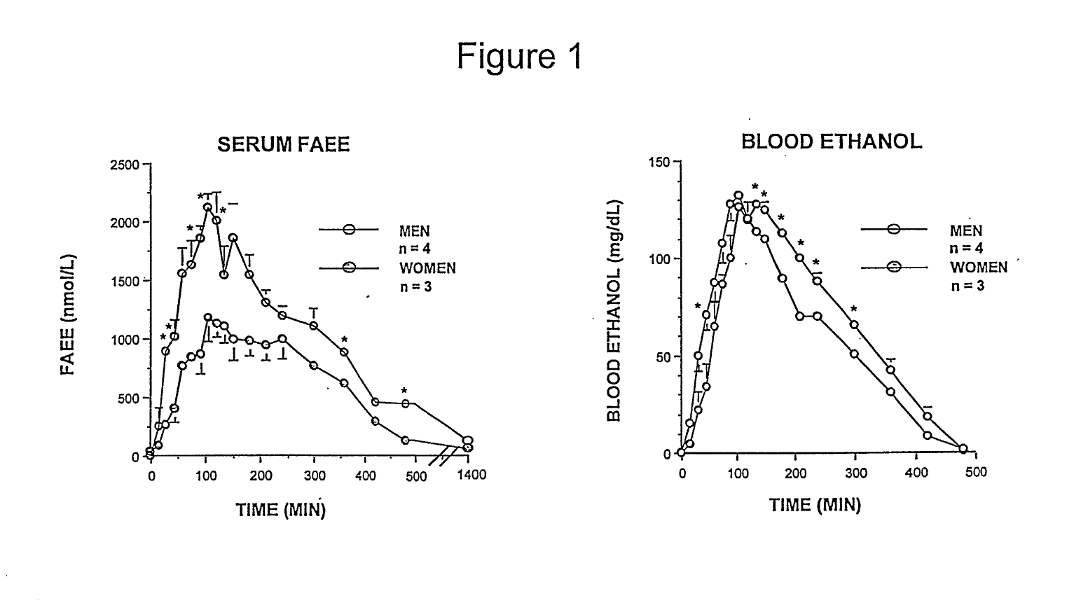

As shown in FIG. 1, the results demonstrate that men have two-fold higher serum FAEE levels than women despite comparable blood ethanol levels. Peak levels of both serum FAEE and blood ethanol were observed between 100 and 150 minutes after the beginning of alcohol intake. The maximum level of blood ethanol was approximately 130 mg/dL for both men and women. The maximum serum FAEE levels were approximately 2100 nmol/L and 1100 nmol/L for men and women, respectively. Blood ethanol decreased to initial levels by about 500 minutes (i.e., 8.3 hours). In contrast, declining levels of serum FAEE levels persisted for 1400 minutes (i.e., 23.3 hours). The decline and persistence of serum FAEE and blood ethanol were similar for men and women.

### Example 2

### Effect of Serum Triglyceride Levels on FAEE Levels

For studies assessing the influence of triglyceride concentrations on FAEE concentrations, 85 residual plasma or serum samples (from 64 men and 21 women) were obtained from the toxicology laboratory at the Massachusetts General Hospital. Plasma or serum was obtained from residual samples positive for blood ethanol in essentially every case, and positive for FAEEs as well. Non-fasting triglyceride (TG) levels were quantitated using a Hitachi 917 automated chemistry analyzer (Boehringer Mannheim Diagnostics).

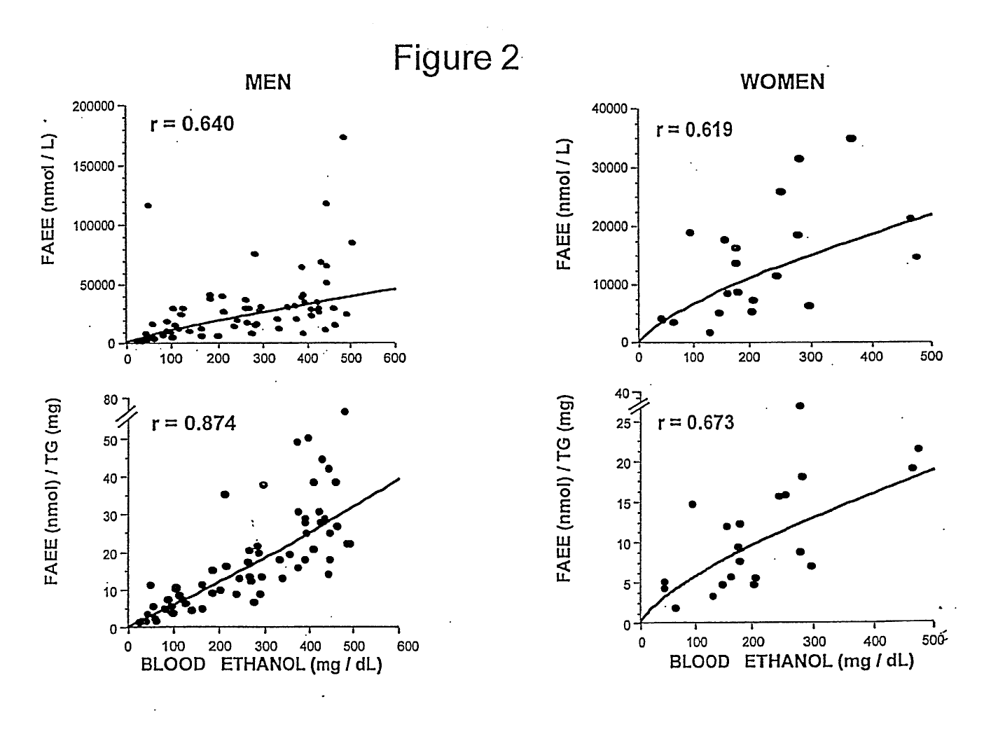

The results shown in FIG. 2 illustrate that the ratio of FAEE levels to triglyceride levels correlates with blood ethanol as does FAEE alone. The relationships of FAEE levels (nmol/L) (top panels) and the ratio of FAEE to TG (nmol/mg) (bottom panels) to blood ethanol levels (mg/dL) are shown. Male and female subjects are shown separately in the left and right panels, respectively. The FAEE/TG ratio appeared linearly related to blood ethanol levels (e.g., r value of 0.874), as did FAEE alone (e.g., r value of 0.640), particularly for the male subjects.

### Example 3

### Effect of Sample Storage on Plasma FAEE Levels

Individual blood samples were collected from multiple subjects known to be at peak ethanol levels. The blood samples were harvested directly into a 10 mL vacutainer tube containing K₃EDTA, and immediately placed on ice. In some embodiments, the tube contains 0.117 mL of 150 g/L K₃EDTA. Tubes were centrifuged at 3420 g at 4° C. for 20 minutes. Following centrifugation, plasma was isolated and frozen at −80° C. prior to analysis. At the time of analysis, samples were thawed, pooled and divided into five 0.5 mL aliquots and stored in conditions in which time and temperature were varied. A control sample was thawed, processed and analyzed immediately.

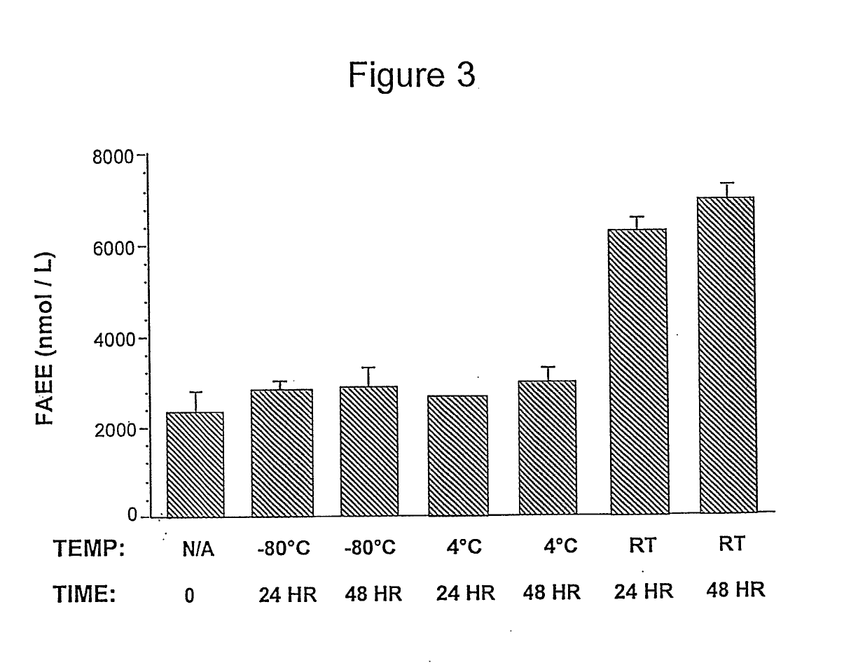

As shown in FIG. 3, there is an artifactual formation of plasma FAEE with storage of samples at room temperature (RT) for 24 hours or more. The control sample contained roughly 2400 nmol/L FAEE. Storage at 4° C. or −80° C. for 24 or 48 hours did not appear to modify this content significantly. Interestingly, incubation at room temperature for 24 hours or more resulted in FAEE levels in excess of those present in the control. It was concluded that in order to monitor levels of FAEE accurately, samples should be maintained at either 4° C. or −80° C. Processing of samples maintained in such a manner can be delayed for 48 hours without significant loss of FAEE.

### Example 4

### Effect of Alcoholic Beverage Type on Plasma FAEE Levels

The effect of alcoholic beverage type on FAEE concentrations was analyzed in eight healthy volunteers (seven women, one man) who were social drinkers. Volunteers were admitted to the General Clinical Research Center (GCRC) on the day of the study. Subjects were required to complete a brief food survey form, recalling the past 24 h of dietary intake, and a drinking history survey (Khavari Alcohol Test). The researchers recorded each subject's weight and height. Volunteers were required to abstain from any ethanol 5 days before and 72 h after the study. Eight subjects were equally divided into 2 groups, one of which drank beer while the other drank vodka. The amount of ethanol that each person received was based on body weight and calculated to increase the blood alcohol level to at least 100 mg/dL. The total ethanol dose was divided into 9 aliquots and consumed over a 90 minute period. Five blood samples were drawn from each subject. One vial (10 mL) of blood was collected before ingestion of ethanol to establish a baseline; the second and third samples were drawn 15 and 30 min after drinking was completed (105 and 120 min after the onset of drinking, respectively). The early time-points were chosen as representative peak ethanol and FAEE levels. Subjects remained in the GCRC until they could be safely discharged, as determined by the blood alcohol concentration and subjective sense of sobriety. Subjects later returned to the GCRC for the 24- and 72-h blood collections. Food was provided during the study by staff dieticians immediately after the 120-min time point. At each time point, blood was collected in 10 mL vacuum tubes containing 0.117 mL of 150 g/L potassium EDTA solution. After sample harvest, the tubes were placed immediately placed on ice. As described above, tubes were centrifuged at 3420 g at 4° C. for 20 minutes and plasma was subsequently isolated. An aliquot of plasma was reserved for ethanol analysis, and another aliquot was used for subsequent FAEE isolation and quantification.

The group which drank beer had FAEE levels of approximately 2400 and 2250 nmol/L at 105 and 120 minutes, respectively. The group which drank vodka had FAEE levels of approximately 2600 and 2200 nmol/L at 105 and 120 minutes, respectively. Thus, there was no significant effect of alcohol type on plasma FAEE levels (nmol/L).

### Example 5

### Effect of the Rate of Ethanol Consumption on Serum FAEE Levels

In studies to determine whether the rate of ethanol intake affects FAEE pharmacokinetics in the blood, six young healthy male Caucasian volunteers were studied. The mean±SE age was 24.2±0.7 years; the mean±SE body weight was 73.4±1.7 kg; the mean±SE body mass index was 22.3±0.3 kg/m²; and the mean±SE lean body mass was 85.8%±1.4% total body weight. All were nonsmokers, and their habitual ethanol intake, based on a representative 1-week dietary recall, was 46±22 g/week. All men had negative clinical histories, and serum aminotransferase, alkaline phosphatase, bilirubin, and albumin concentrations within the appropriate reference intervals, and none had any serological evidence of viral hepatitis.

The subjects fasted for at least 12 h before the study. At the initiation of the study, subjects were placed on a bed in a semirecumbent position, and an indwelling catheter (kept open with normal saline) was inserted into an antecubital vein for blood sampling. After 1 h of baseline measurements (three blood drawings spaced exactly 20 min apart), the subjects were given 31.9±0.6 g of ethanol (0.43±0.004 g/kg body weight) to ingest in a 2-min oral bolus. The subjects ingested the ethanol as a 10% volume solution (diluted with tap water only), containing 2 drops of a concentrated flavored extract used for baking. Venous blood was then sampled at 20-min intervals over a total period of 5 h. Blood ethanol concentrations were determined by GC, using head space injection. Serum was isolated from blood by centrifugation at 2800 g for 10 min at 4° C. and frozen immediately at −70° C. for subsequent measurement of FAEEs.

Subjects in the slow rate of consumption were given 100-proof vodka mixed with fruit juice in a 1:3 ratio which was weight-adjusted to increase blood ethanol levels to approximately 120-147 mg/dL. The beverage was divided in 9 equal aliquots which the subjects drank over a 90 minute period, drinking 1 aliquot every 10 minutes. Blood was collected from the peripheral venous catheter into vacutainer tubes without anti-coagulant for serum FAEE, and with the anti-coagulant sodium citrate for plasma ethanol.

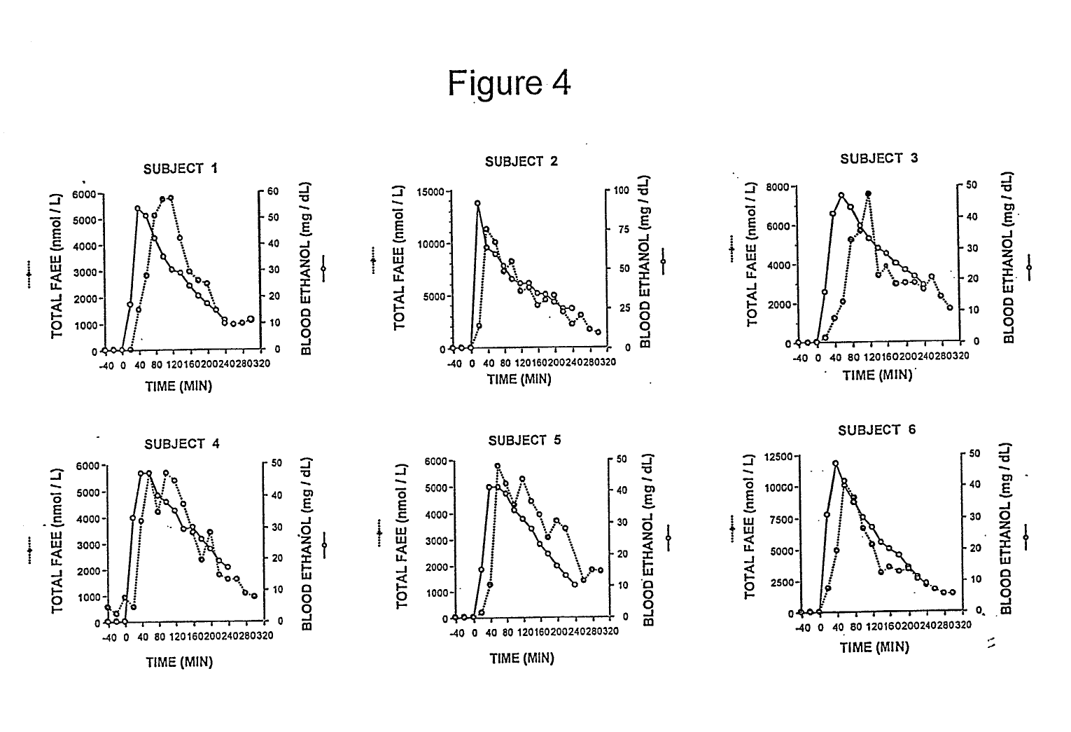

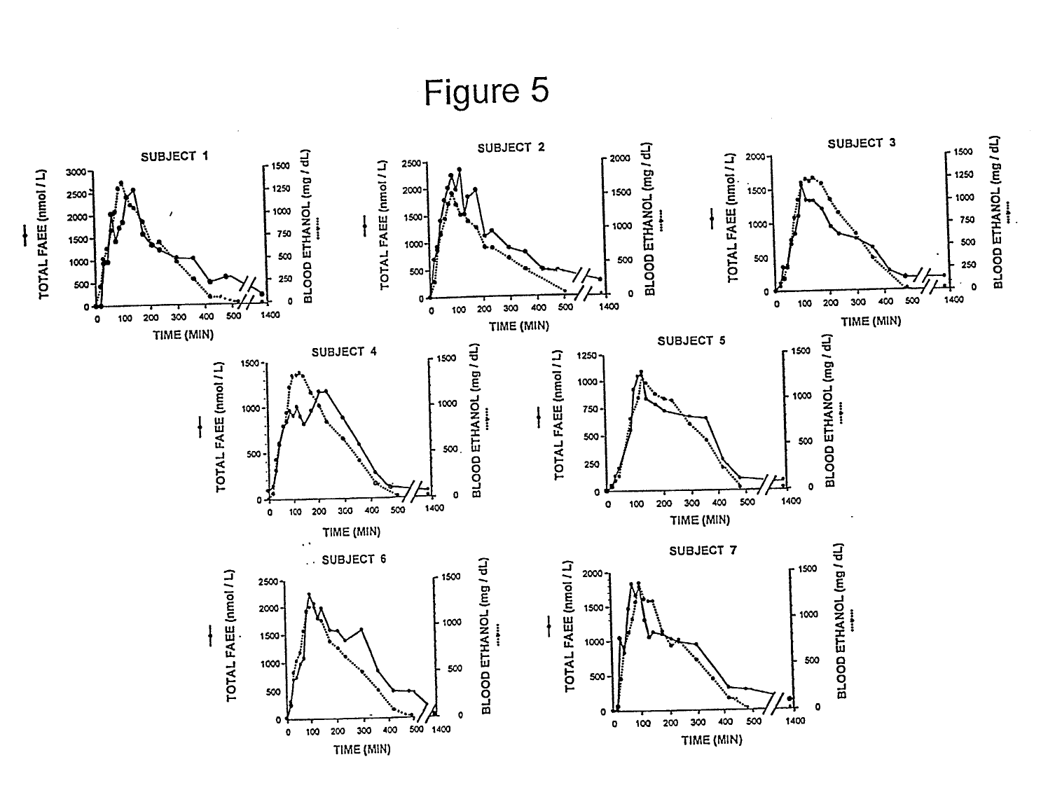

FIGS. 4 and 5 show the kinetics of increase and decrease of total FAEE and blood ethanol levels resulting from a rapid rate (FIG. 4) and a slow rate (FIG. 5) of alcohol consumption. The rate of ethanol ingestion had no effect on total serum FAEE levels in the subjects tested.

### Example 6

### Distribution of Plasma FAEE Species in Chronic Alcoholics vs. Acutely Intoxicated Individuals

Plasma was obtained from chronic alcoholics (i.e. chronic drinkers) at the time of admission to a detoxification program. Acutely intoxicated control subjects used for comparison in this study were normal, healthy volunteers who also participated in the study concerning gender and alcoholic beverage type (see above).

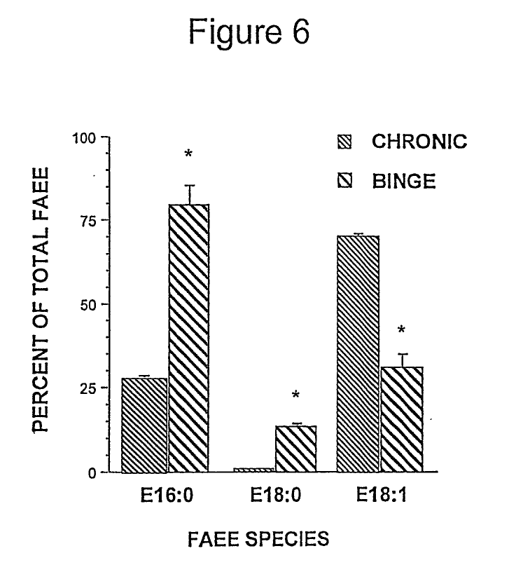

FIG. 6 is a histogram showing the percent of total FAEE which is either ethyl palmitate (i.e., E16:0), ethyl stearate (i.e., E18:0), or ethyl oleate (i.e., E18:1), in acute and chronic drinkers. The data are presented as means (±SEM) each for the group of chronic and acute (i.e., binge) drinkers. The proportion of total FAEE which is ethyl palmitate (i.e., E16:0) was almost three times higher in acute drinkers than in chronic drinkers. Ethyl palmitate was also the most prevalent of the three types of FAEE in acute alcoholics. Ethyl stearate is the least prevalent type of FAEE in both chronic and acute, although the proportion of FAEE which it represents is almost 15-fold higher in acute drinkers as compared to chronic drinkers. Chronic drinkers demonstrated the highest proportion of ethyl oleate. Ethyl oleate represented almost 75% of total FAEE in chronic drinkers as compared to about 30% in acute drinkers. Thus, chronic alcoholics could be distinguished from acutely intoxicated individuals based on the prevalence of FAEE species in their plasma, with chronic drinkers having a higher percentage of ethyl oleate and a lower percentage of ethyl palmitate, and acute drinkers having a higher percentage of ethyl palmitate and a lower percentage of ethyl oleate. Statistical analysis showed that the differences between the two groups were significant, at p<0.01, for each FAEE species tested. The distribution of plasma FAEE species in acute drinkers is different from that of chronic drinkers.

### Example 7

### Analysis of FAEE Levels in Acute and Chronic Drinkers at Peak Ethanol Levels and 24 Hours After Peak Levels

A subset of human subjects presenting at the Massachusetts General Hospital with apparent alcohol related symptoms were entered into this study. Blood was collected from such subjects immediately at the time of presentation, which was assumed to be at peak or near peak ethanol levels, and again 24 hours after presentation. A medical history taken from each subject was used to categorize the subject as a chronic alcoholic or an acute (i.e., binge) drinker. Blood samples were processed, as described herein, to determine the absolute levels of ethyl palmitate, ethyl oleate and total FAEE in plasma.

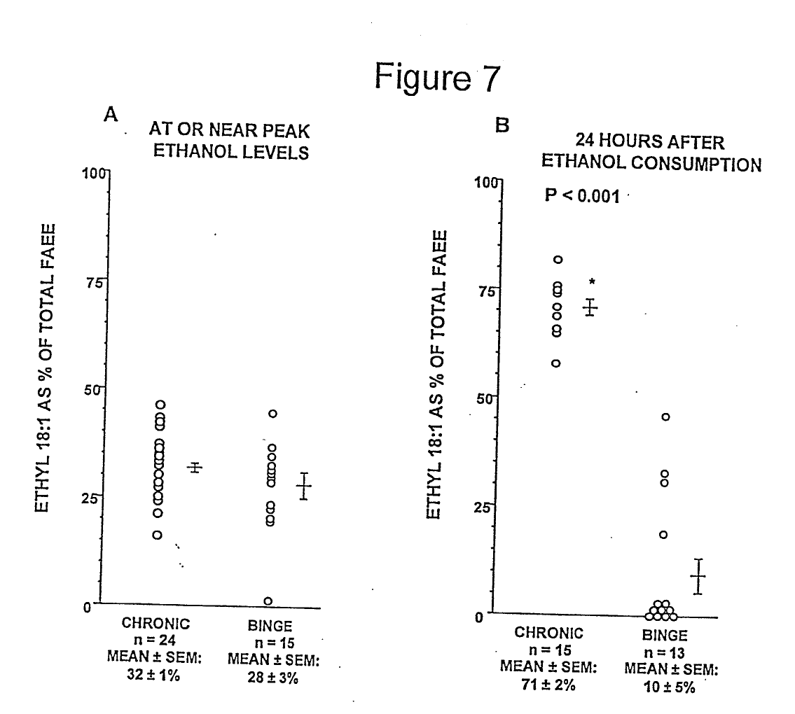

FIG. 7 shows the proportion of total FAEE which is ethyl oleate in the plasma of chronic and binge drinkers at or near peak alcohol levels and 24 hours after peak alcohol levels. The ranges and means for these groups is shown in Table 1. At times corresponding to peak or near peak ethanol levels, much overlap existed between chronic and binge drinkers, with binge drinkers on average having only slightly higher proportions of ethyl oleate (i.e., 32% vs. 28%). However in samples drawn at 24 hours after peak ethanol levels, chronic drinkers had a significantly greater proportion of ethyl oleate (i.e., 71%) as compared to binge drinkers (i.e., 10%). This difference was statistically significant at p<0.001. This demonstrated that proportion of ethyl oleate, 24 hours after peak ethanol levels, is a strong discriminator for chronic and binge drinkers.

Table 1

| | Proportion of | Proportion of | |
|---|---|---|---|
| | Ethyl Oleate in | Ethyl Oleate in | Threshold for |
| | Chronic Drinkers | Binge Drinkers | Positivity of |
| | (Mean +/− SEM) | (Mean +/− SEM) | Chronic |
| Peak or Near | 0.16-0.46 | 0.01-0.44 | N/A |
| Peak | (0.32 +/− 0.02) | (0.28 +/− 0.02) | |
| 24 Hours After | 0.58-0.82 | 0.01-0.46 | >0.52 |
| Peak | (0.71 +/− 0.02) | (0.09 +/− 0.04) | |

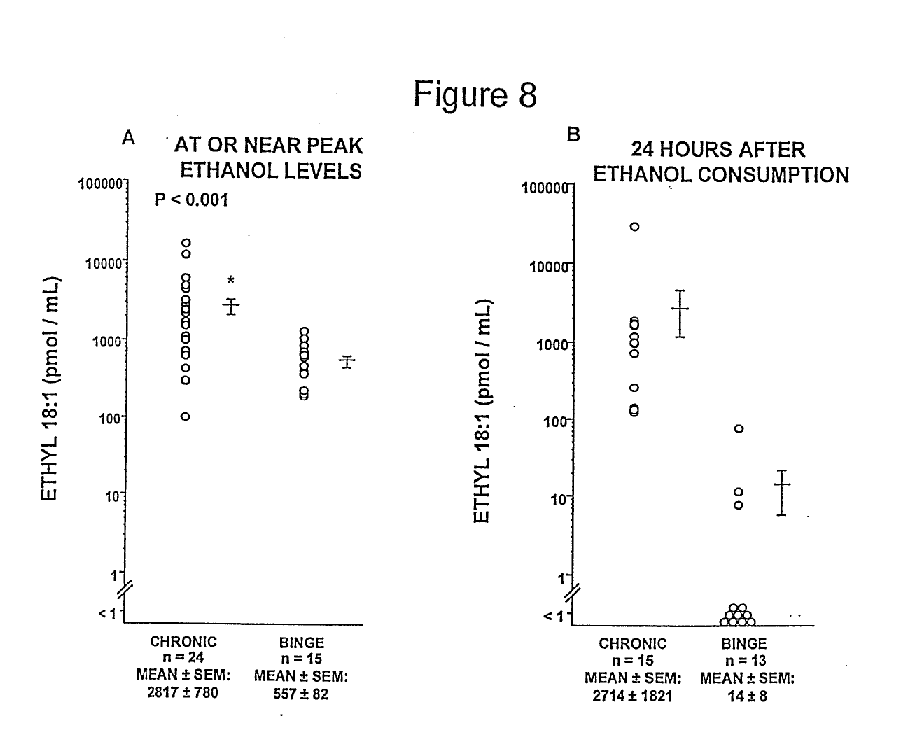

Ethyl oleate concentrations are shown in FIG. 8, with corresponding ranges and means listed in Table 2. Ethyl oleate concentrations at or near peak ethanol levels were useful for distinguishing only a subset of chronic alcoholics from binge drinkers (e.g., those with >1277 pmol/mL of ethyl oleate). At 24 hours after peak ethanol levels, however, the distinction between chronic and binge drinkers using this parameter was more clear. Concentrations of ethyl oleate greater than 100 pmol/mL were indicative of chronic drinkers. The majority of binge drinkers (i.e., 9 out of 13) had negligent concentrations (i.e., <1 pmol/mL) of ethyl oleate.

TABLE 2

| | Concentration of | Concentration of | |
|---|---|---|---|
| | Ethyl Oleate in | Ethyl Oleate in | Threshold for |
| | Chronic Drinkers | Binge Drinkers | Positivity |
| | (Mean +/− SEM) | (Mean +/− SEM) | Chronic |
| | (pmol/mL) | (pmol/mL) | (pmol/mL) |
| Peak or Near | 96-16011 | 184-1049 | >1277 |
| Peak | (2817 +/− 780) | (557 ± 82) | |
| 24 Hours After | 120-28109 | 0-80 | >100 |
| Peak | (2714 ± 1821) | (14 ± 8) | |

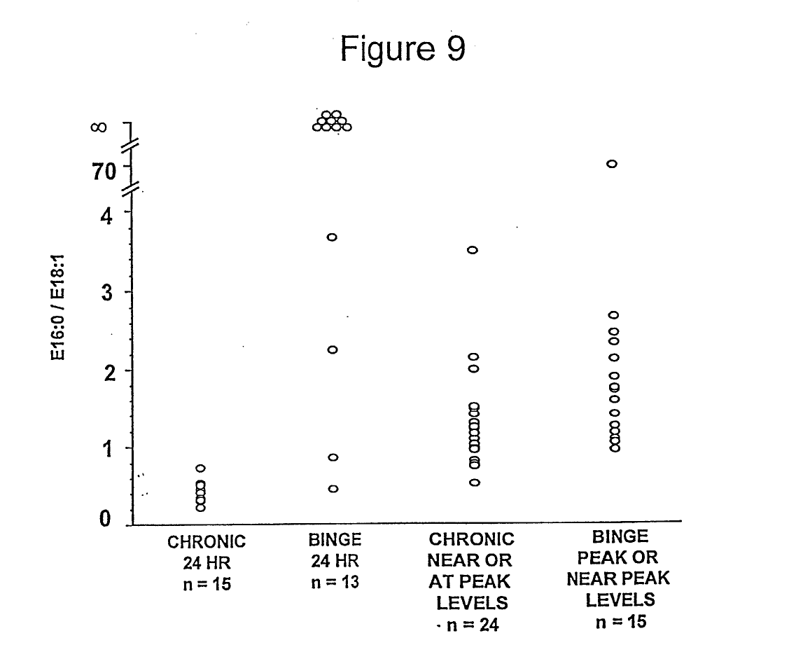

The ratio of ethyl palmitate to ethyl oleate (i.e., the P/O ratio) is shown in FIG. 9 as a function of time after alcohol ingestion for chronic and acute drinkers. Table 3 summarizes the ranges and averages for the groups shown in FIG. 9. At times of peak or near peak alcohol levels, the ranges of P/O ratios for chronic and acute drinkers overlapped significantly. A day later, however, most binge drinkers had P/O ratios approximating ∞, while all chronic drinkers had P/O ratios of less than 1.0. Only two of the thirteen binge drinkers had P/O ratios within the chronic drinker range.

TABLE 3

| | Ethyl Palmitate to | Ethyl Palmitate to | |
|---|---|---|---|
| | Ethyl Oleate Ratio | Ethyl Oleate Ratio | Threshold for |
| | in Chronic Drinkers | in Binge Drinkers | Positivity of |
| | (Mean +/− SEM) | (Mean +/− SEM) | Chronic |
| Peak or | 0.53-3.49 | 0.96-70.65 | N/A |
| Near Peak | (1.27 +/− 0.12) | (6.28 +/− 5.0) | |
| 24 Hours | 0.22-0.73 | 0.46-∞ | >0.9 |
| After Peak | (0.41 +/− 0.03) | (69.8 +/− 13.1)* | |

*Mean was calculated using a value of 100 for maximum points (i.e., where the ratio was in fact ∞.)

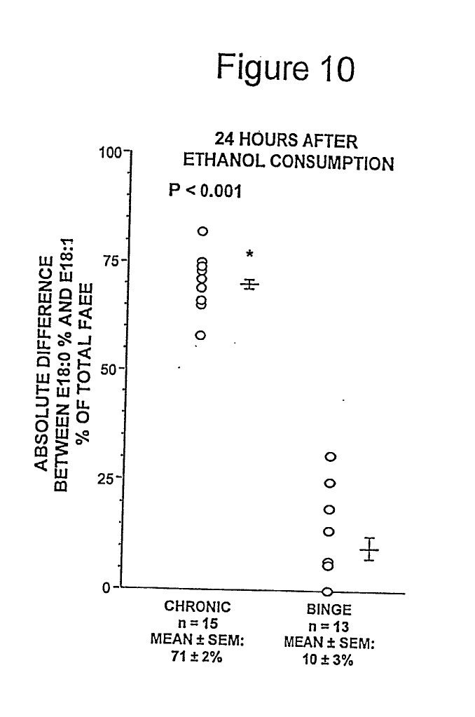

FIG. 10 shows the difference between the proportion of total FAEE which is ethyl stearate (E18:0) and the proportion of total FAEE which is ethyl oleate (E18: 1) in samples harvested from chronic alcoholics and binge drinkers 24 hours after alcohol consumption. The data suggest that binge drinkers have similar proportions of ethyl stearate and ethyl oleate while chronic drinkers have a much higher concentration of ethyl stearate than ethyl palmitate in their blood. The mean difference for chronic alcoholics is roughly 70% while the mean difference in binge drinkers is about 10%. This difference is statistically significant at p<0.001. Thus, chronic alcoholics and binge drinkers can also be distinguished on the basis of the difference between the proportion of total FAEE which is ethyl stearate and the proportion of total FAEE which is ethyl oleate. A difference greater than or equal to 45 is indicative of a chronic alcoholic. Conversely, a difference less than 45 is indicative of a binge drinker. This parameter can be used alone or in combination with the other parameters described herein to identify chronic alcoholics or binge drinkers alike.

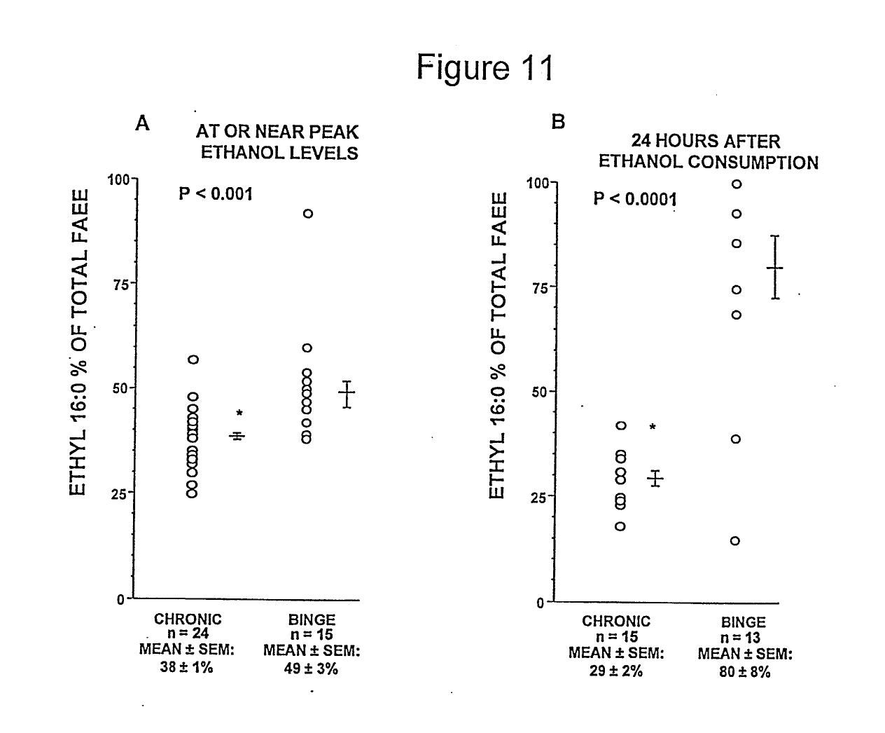

FIG. 11 shows the proportion of total FAEE which is ethyl palmitate relative to time after peak or near peak ethanol levels for binge and chronic drinkers. At peak or near peak alcohol levels, the mean proportion of total FAEE which is ethyl palmitate in chronic alcoholics is 38% while for binge drinkers it is 49%. At 24 hours after peak ethanol levels, ethyl palmitate represents, on average, 29% of total FAEE in chronic alcoholics and 80% of total FAEE in binge drinkers. This difference is statistically significant at p<0.0001. When used at 24 hours after peak ethanol levels, this parameter may be used to distinguish between chronic and binge drinkers. Chronic drinkers can be identified as persons with a proportion of total FAEE which is ethyl palmitate less than 42%. Conversely, binge drinkers are identified on the basis of ethyl palmitate levels exceeding 42% of total FAEE. Preferably, this parameter is used in combination with another discriminator to distinguish between chronic and binge drinkers.

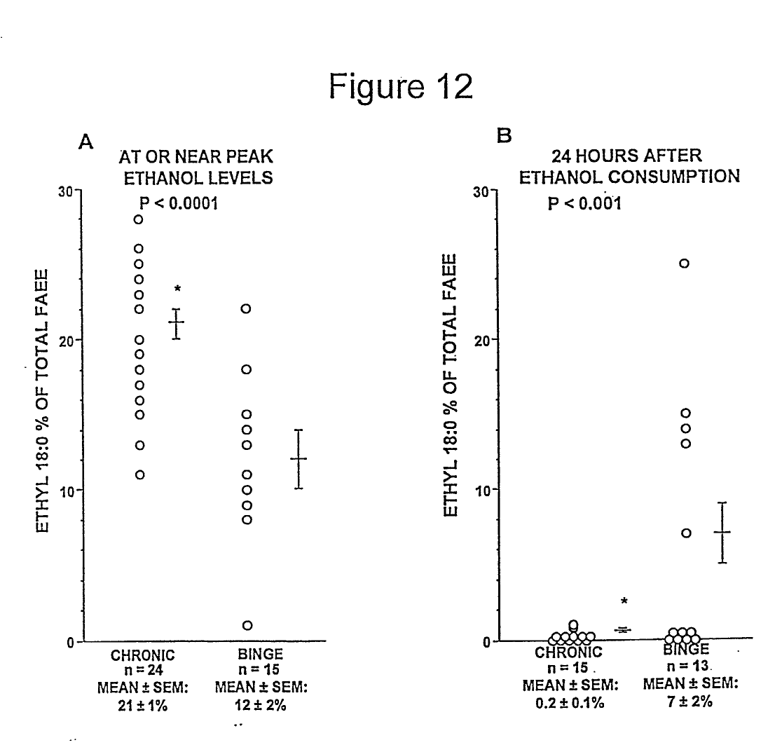

FIG. 12 demonstrates the proportion of total FAEE which is ethyl stearate as a function of time relative to peak ethanol levels in chronic and binge drinkers. At or near peak ethanol levels, considerable overlap exists between chronic alcoholics and binge drinkers. At 24 hours after ethanol consumption, binge drinkers have a higher proportion of ethyl palmitate in their blood than do chronic alcoholics. However, even at these time points, considerable overlap exists between the individual samples.

Taken together, these data prove that it is possible to distinguish between chronic alcoholics and binge drinkers based on any three of these parameters (i.e., proportion of ethyl oleate in plasma, concentration of ethyl oleate in plasma and P/O ratio). Any single parameter can be used for the determination, or some combination of the three (e.g., either two or three) can be used as well. Moreover, these measurements are more robust as discriminators if these are used at the 24 hour time-point following peak alcohol levels.

### Example 8

### Analysis of FAEE Content Ante-Mortem in Liver and Adipose Tissue Following Alcohol Introduction

In one arm of this study, rats were used as subjects. Ten male rats were used, each weighing between 225 and 250 grams. Six of these were injected intraperitoneally with 2 g/kg of absolute ethanol, diluted to 50% with normal saline. The remaining 4 rats were injected with normal saline alone. All rats were sacrificed 2 hours after injection by carbon dioxide asphyxiation, and then placed in a small box outdoors for 5 days at temperatures of 30° F. to 40° F.

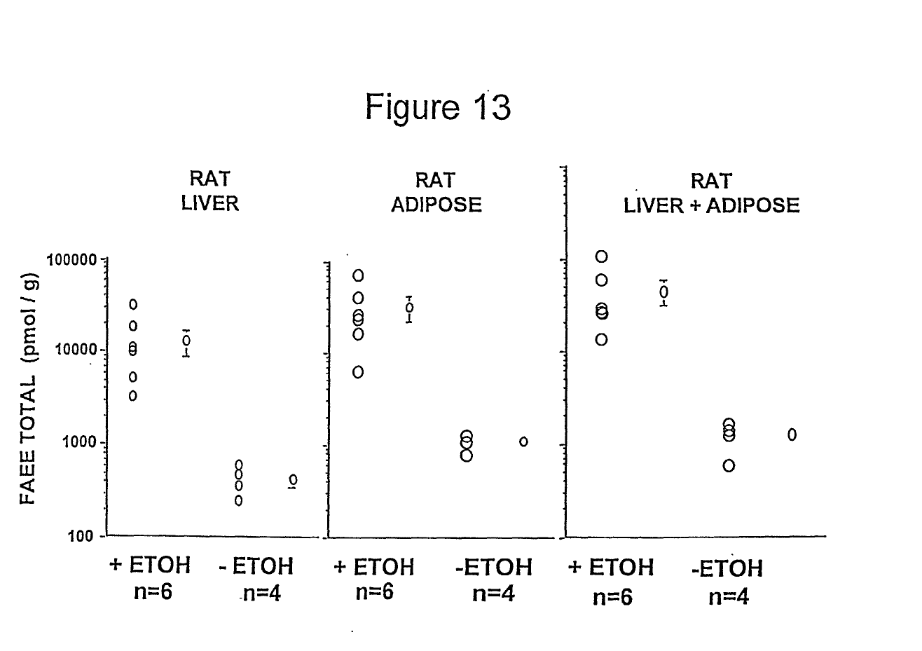

FIG. 13 shows the amount of total FAEE (pmol/g) in liver tissue (a), adipose tissue (b), and in both liver and adipose tissue combined (c), for rats injected with alcohol (+ETOH) and rats receiving only saline (−ETOH). In all three measurements, the total FAEE levels in rats receiving ethanol were significantly greater than control rats. In liver tissue, total FAEE levels ranged from 3000 to 35000 pmol/g in alcohol injected rats. Saline injected mice had FAEE levels in the range of 250-600 pmol/g in liver tissue. Liver of alcohol injected rats contained, on average, almost 33-fold more total FAEE than control livers. FAEE content in adipose tissue of alcohol injected rats ranged from 6500-80000 pmol/g, while in control rats, the range was 900-1500 pmol/g. Thus a similar difference between alcohol and saline injected rats was observed for the average amount of total FAEE in adipose tissue as in liver. The sum of total FAEE in liver and adipose tissue was also able to discriminate between the groups of rats, and importantly, using this combined measurement, there existed almost an order of magnitude difference between individual rats in different groups. Table 4 summarizes these results.

TABLE 4

| | FAEE Range in Saline | FAEE Range in | Threshold |
|---|---|---|---|
| | Injected Rats | Alcohol Injected Rats | for |
| | (Mean +/− SEM) | (Mean +/− SEM) | Positivity |
| | (pmol/g) | (pmol/g) | (pmol/g) |
| Liver | 250-595 | 3074-30602 | >1835 |
| | (420 +/− 74) | (12867 +/− 4118) | |
| Adipose | 0-1302 | 6524-74806 | >3913 |
| | (1075 +/− 140) | (31581 +/− 9816) | |
| Liver and | 594-1660 | 16332-105409 | >8996 |
| Adipose | (1226 +/− 225) | (44449 +/− 13512) | |

In the second arm of this study, human subjects were used. FAEEs were extracted from the liver and adipose tissue of humans, post-mortem. Fifteen post-mortem cases were used from the Massachusetts Medical Examiner's Office and the Department of Pathology at the Massachusetts General Hospital. Blood ethanol was also quantitated from autopsy specimens on all cases. Three out of 11 were positive for blood ethanol. The aim of this study was to determine if any difference existed in the total FAEE content in liver and adipose tissue in the post-mortem human subjects with detectable versus undetectable blood ethanol.

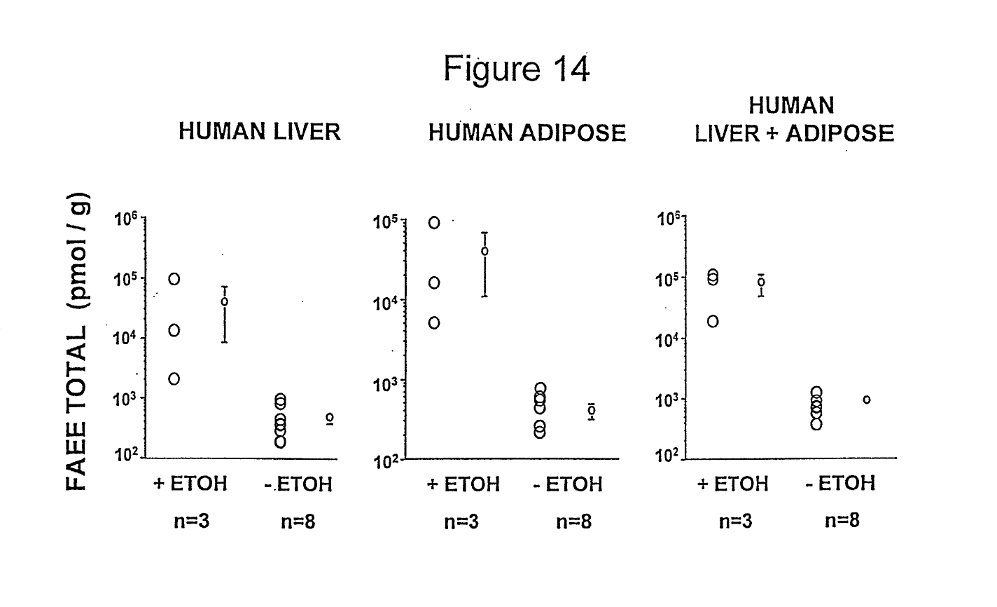

FIG. 14 demonstrates the total FAEE content of liver (a), adipose tissue (b), and combined liver and adipose tissue (c) in human post-mortem subjects with (+ETOH) and without (−ETOH) detectable blood ethanol levels. The ranges of FAEE content for each group are shown in Table 5. As was observed in the rat study, total FAEE content of both liver and adipose tissue was significantly higher in alcohol positive individuals than alcohol negative individuals. A greater difference between the two groups was observed in adipose tissue than in liver alone, however the best discriminator was the combined FAEE content of the liver and adipose tissue. The recommended protocol for FAEE quantitation from human liver and adipose tissue as an indicator of ante-mortem ethanol intake requires the harvest of at least 5 grams each of liver and adipose tissue from the panniculus, followed by the extraction and quantitation of FAEE as described herein.

TABLE 5

| | FAEE Range in | FAEE Range in | |
|---|---|---|---|
| | Ethanol Negative | Ethanol Positive | |
| | Humans | Humans | Threshold for |
| | (Mean +/− SEM) | (Mean +/− SEM) | Positivity |
| | (pmol/g) | (pmol/g) | (pmol/g) |
| Liver | 193-1034 | 2226-100062 | >1630 |
| | (482 +/− 159) | (38936 +/− 30768) | |
| Adipose | 0-787 | 5315-94983 | >3051 |
| | (402 +/− 90) | (39047 +/− 28165) | |
| Liver and | 402-1308 | 19836-116906 | >10572 |
| Adipose | (884 +/− 107) | (77984 +/− 29625) | |

On the basis of studies in both humans and rats, FAEE concentration in liver alone, or the combined total FAEE concentration in liver and adipose tissue combined, are both useful markers of ante-mortem ethanol intake. The combined FAEE concentration in liver and adipose tissue combined is a preferred measure of alcohol intake given its ability to distinguish clearly between subjects who consumed alcohol prior to death and those that did not.

### Example 9

### Other Post-Mortem Markers of Pre-Mortem Ethanol Intake

This study was approved by both the Massachusetts Medical Examiner Office Committee and the Massachusetts General Hospital Pathology Quality Assurance Committee. Twenty-four sets of samples were collected at the Massachusetts State Medical Examiner's Office and 7 sets of samples were obtained from the Pathology Department of Massachusetts General Hospital. Medical history, history of ethanol ingestion (obtained from the organ bank, treating physician, health insurance records, and/or relatives), and the blood ethanol level at autopsy were obtained in each case. Only cases with available blood ethanol levels were included in this study. Individuals who had detectable blood ethanol levels at the time of autopsy, with or without a history of ethanol abuse, were grouped as “positive for ethanol.” Individuals categorized in the chronic alcoholic or social drinker groups met all of the following criteria a) no known alcohol intake immediately prior to death; b) undetectable blood ethanol level in blood samples collected at autopsy; and c) no suspicious incident at the time of death. In addition, chronic alcoholics were diagnosed according to the Diagnostic and Statistical Manual-IV.

Samples of liver and adipose tissue were collected at autopsy from randomly selected cases and FAEE were isolated and quantitated from these organs as mass per gram wet weight. Postmortem analysis of blood involved assessment for ethanol and other drugs. Immediately upon arrival in the laboratory, samples were stored at −80° C. until FAEE analysis was performed. The postmortem interval between death and autopsy ranged from 5 to 29 hours, with a mean of 16 hours.

A portion of each tissue was harvested, weighed, and immediately placed on ice, then homogenized (1:10, w/v) in protease inhibitor buffer containing 10 mM N-2-hydroxyethylpiperazine-N′-2-ethanesulfonic acid, 20 μg/mL phenylmethylsulfonyl fluoride, 1 mM benzamidine, and 0.01% soybean trypsin inhibitor (pH 7.34) using a Fisher PowerGen 125 Homogenizer (Fisher Scientific, Pittsburgh, Pa.) equipped with a 10×195 mm sawtooth generator. Two mL of cold acetone were added to 1 mL of the homogenate, which was kept on ice. The sample was then vortexed for 1 min and centrifuged for 5 min at 170×g at 4° C., and the supernatant was transferred to a separate tube. An internal standard of 500 pmol ethyl heptadecanoate (E 17:0) (Nu Chek Prep, Elysian, Minn.) was added to each sample along with 6 mL of hexane. The mixture was vortexed for 1 min and centrifuged at 170×g for 5 min at 4° C. The hexane layer was transferred to a separate tube and the aqueous phase re-extracted with an additional 2 mL of hexane. The wash was pooled with the original hexane layer, evaporated to dryness under nitrogen and resuspended in 200 μL of hexane. FAEEs were isolated from the lipid extract using solid-phase extraction (SPE). Briefly, aminopropyl columns (Bond-Elut LRC, Varian Diagnostics, Harbor City, Calif.) were placed on a Vac-Elut vacuum apparatus (Varian Diagnostics, Harbor City, Calif.) set at 10 kPa. The columns were pre-conditioned with 4 mL of hexane, followed by 4 mL of dichloromethane. The 200 mL aliquot of lipid extract was then applied, and FAEEs were eluted from the column with an additional 4 mL of hexane and 4 mL of dichloromethane. The eluate was next evaporated to a volume of 50 μL and a 1 μL aliquot was injected into a Hewlett-Packard 5971 mass spectrometer (GC-MS) equipped with Supelcowax 10 capillary column (Supelco, Inc., Bellefonte, Pa.). The injector and detector were maintained at 260° C. and 280° C., respectively. The oven program was initially maintained at 150° C. for 2 min, then ramped to 200° C. at 1° C./min for 4 min, ramped again at 5° C./min to 240° C. and held for 3 min, and finally ramped to 270° C. at 10° C./min and held for 5 min. Carrier gas flow rate was maintained at a constant 0.75 mL/min throughout. Single ion monitoring was performed, quantitating appropriate base ions for individual fatty acid ethyl esters (i.e., ions 67, 88 and 101 for ethyl palmitate (E16:0), ethyl heptadecanoate (E17:0), ethyl stearate (E18:0), ethyl oleate (E18:1), and ethyl linoleate (E18:2); and ions 79, 91 and 117 for ethyl arachidonate (E20:4), ethyl eicosapentaenoate (E20:5) and ethyl docosahexaenoate (E22:6). FAEE quantitation was determined by interpolation of the slope of the standard curve, generated by plotting FAEE/E17:0 peak height ratios to concentration ratios. Total FAEE mass was determined by addition of masses of individual FAEE (E16:0-E22:6).

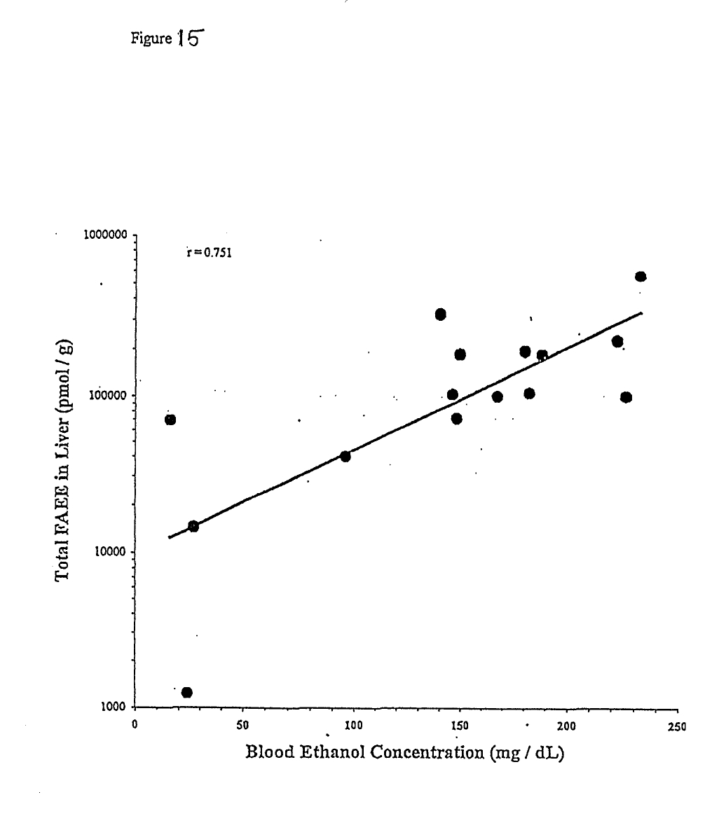

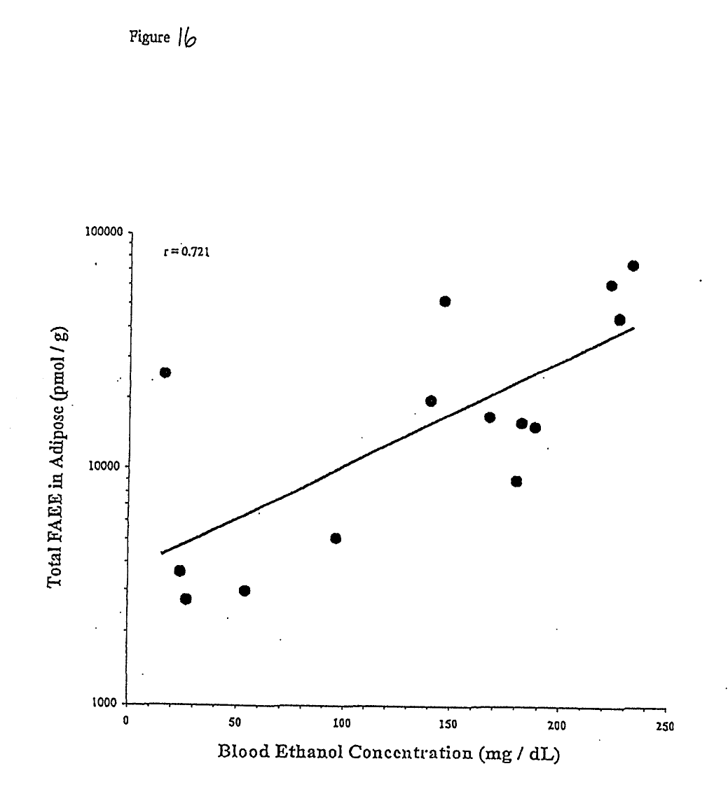

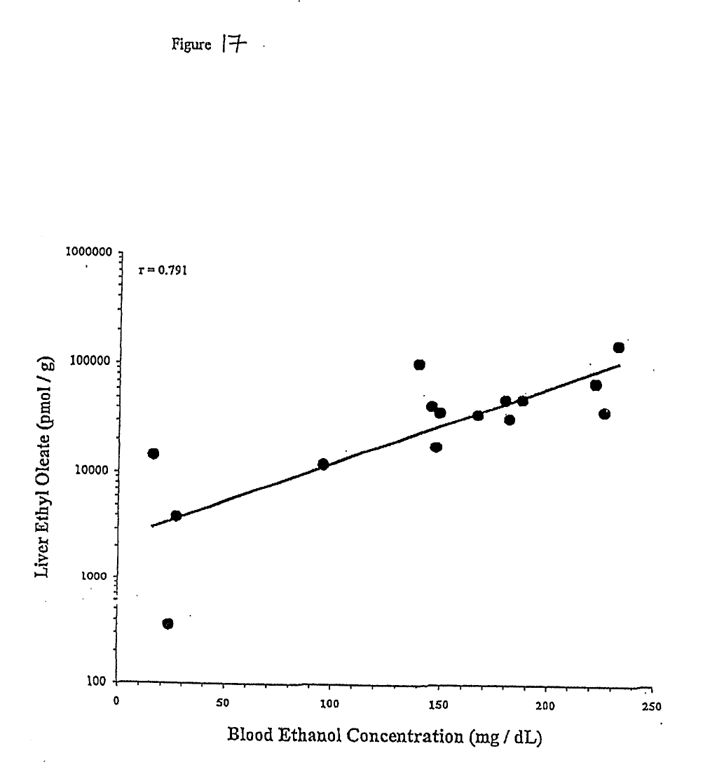

Subjects with detectable blood ethanol at the time of autopsy had extremely high levels of FAEE in the liver and in the adipose tissue. The levels in the liver nearly always exceeded the levels present in the adipose tissue in this group. The social drinkers had the lowest levels of the FAEE in both liver and adipose tissue. FIG. 15 shows the correlation of blood ethanol concentration to FAEE mass in the liver. There was a correlation between blood ethanol concentration and total FAEE in adipose tissue (FIG. 16). One particular fatty acid ethyl ester in the liver, ethyl oleate, showed an even higher correlation with the blood ethanol concentration (FIG. 17), with an r-value of 0.791.

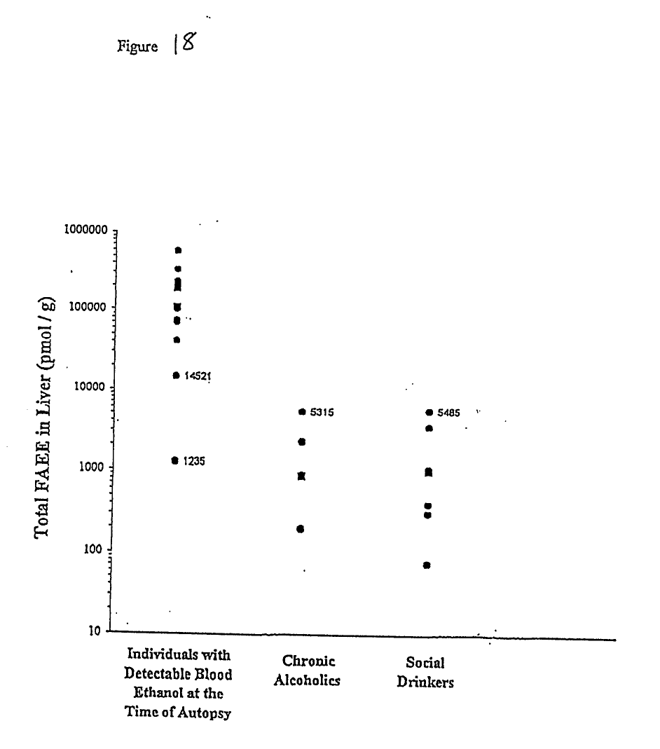

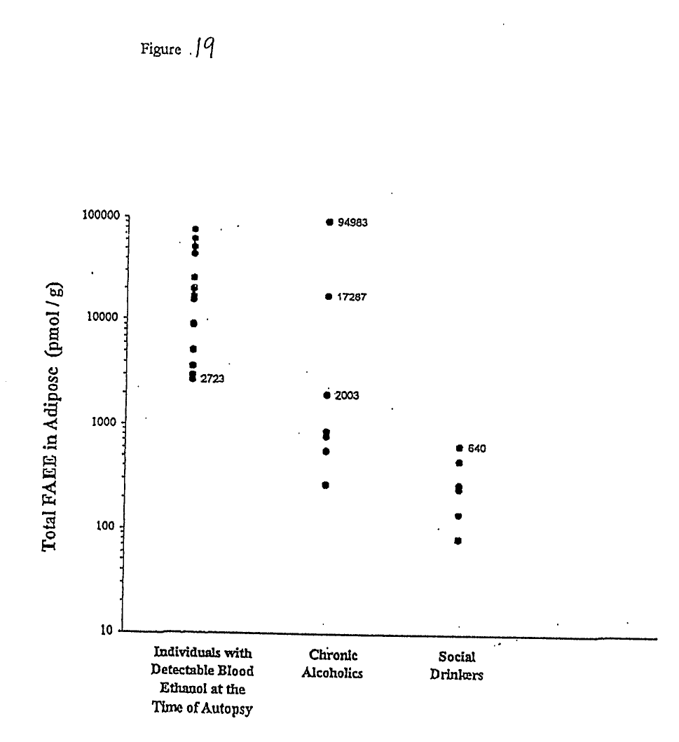

FIG. 18 shows the individuals in each of the groups and illustrates that, with the exception of the subject whose liver FAEE/adipose FAEE ratio was below 2, there was a complete separation in total FAEE between the individuals with detectable blood ethanol at the time of autopsy and the chronic alcoholics and social drinkers. This indicates that the FAEE in the liver is an effective marker in a postmortem setting for pre-mortem ethanol intake. The highest value for total FAEE in liver for chronic alcoholics and for social drinkers was 5,485 pmol/g, and the lowest value for the individuals with detectable blood ethanol at the time of autopsy, other than the one outlier, was 14,521 pmol/g. This is approximately 3-fold difference between the highest level in the chronic alcoholics and social drinkers versus the lowest level in the individuals with detectable blood ethanol at the time of death. FIG. 19 shows the individual points for the FAEE in adipose tissue. FAEE in the adipose tissue of social drinkers was well below that in individuals with detectable blood ethanol at time of autopsy.

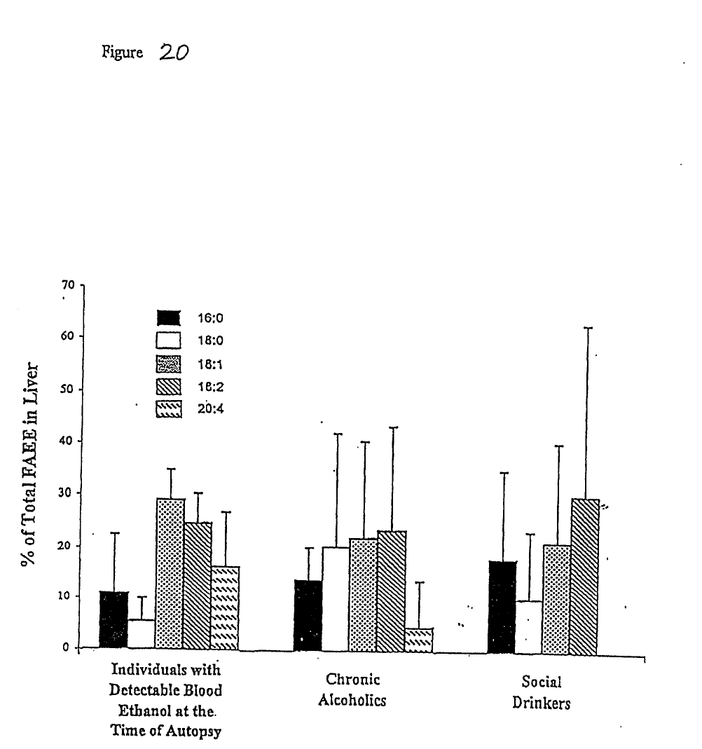

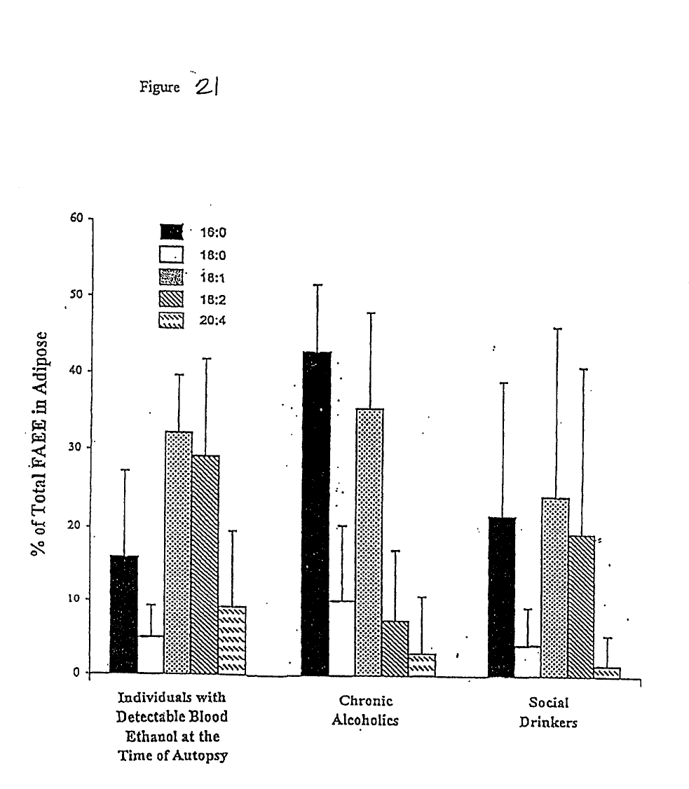

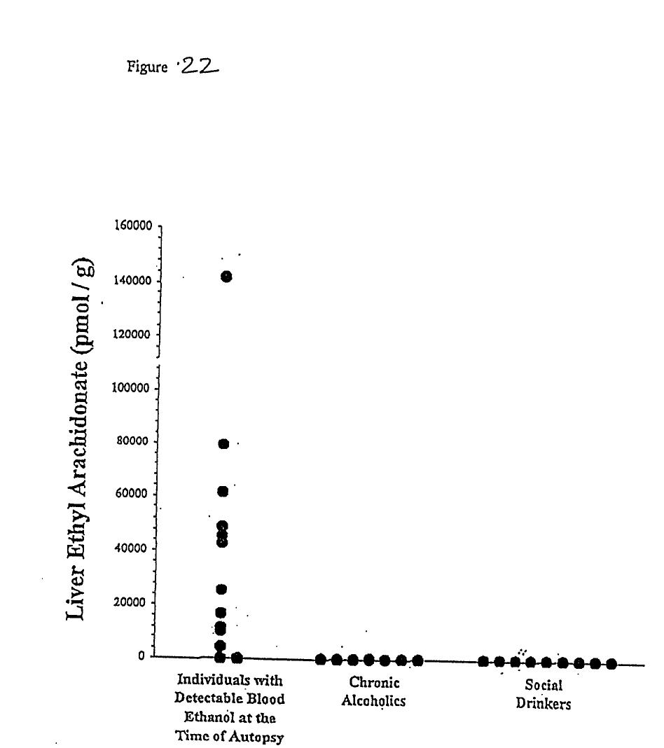

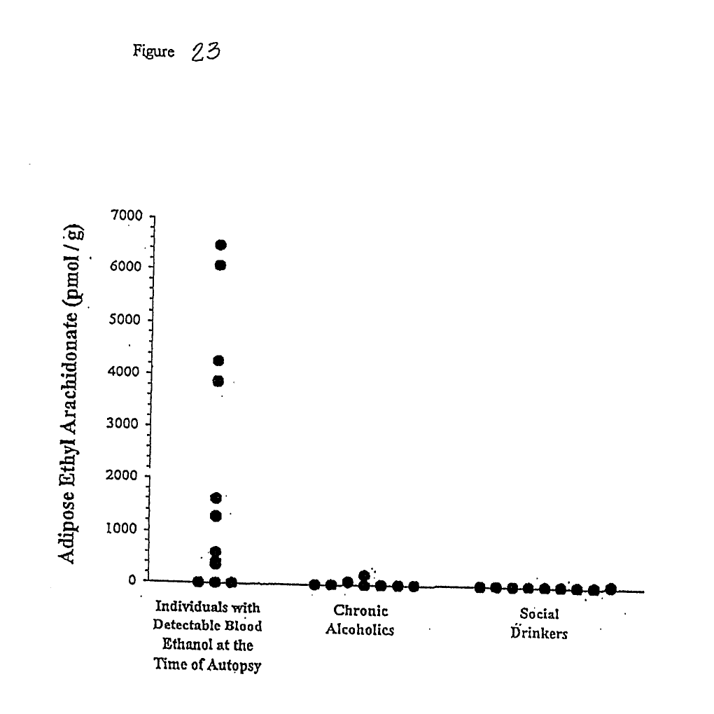

FIG. 20 shows the distribution of the fatty acids in the FAEE from the liver, and FIG. 21 shows the fatty acid composition of the FAEE in adipose. Ethyl arachidonate in both liver and adipose was markedly higher in individuals with detectable blood ethanol at the time of autopsy than it was in chronic alcoholics and social drinkers. Data in FIGS. 22 and 23 show the individual cases relative to ethyl arachidonate levels. The presence of ethyl arachidonate at any concentration in both liver and adipose was also able to differentiate individuals with detectable blood ethanol at the time of autopsy, again with the exception of the one “outlier” who had no liver or adipose ethyl arachidonate.

The results of this study indicate that the total FAEE in liver and adipose, the ratio of liver FAEE/adipose FAEE, and the presence of ethyl arachidonate in both liver and adipose are all useful autopsy markers of pre-mortem ethanol intake.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

I claim:

1. A method for identifying a chronic alcoholic comprising
determining a concentration of ethyl oleate (O) and a concentration of ethyl palmitate (P) in a sample from a subject, and
determining a P/O ratio of the concentration of ethyl palmitate (P) to the concentration of ethyl oleate (O) in the sample,
wherein a P/O ratio less than 0.9 is indicative of a chronic alcoholic.

2. The method of claim 1, further comprising identifying a chronic alcoholic based on
determining a concentration of ethyl oleate in a sample from the subject,
wherein a concentration of ethyl oleate greater than 100 pmol/mL in the sample is indicative of a chronic alcoholic.

3. The method of claim 1 or 2, further comprising identifying a chronic alcoholic based on
determining a concentration of total FAEE (T) in the sample and
determining an O/T ratio of the concentration of ethyl oleate (O) to the concentration of total FAEE (T) in the sample,
wherein an O/T ratio greater than 0.52 is indicative of a chronic alcoholic.

4. The method of claim 1, wherein the sample is blood.

5. The method of claim 1, wherein the sample is isolated from the subject within 4 days of the cessation of alcohol intake.

6. The method of claim 1, wherein the sample is isolated from the subject within 24 hours of the cessation of alcohol intake.

7. The method of claim 1, wherein the sample is isolated from the subject within 12 hours of the cessation of alcohol intake.

8. The method of claim 1, wherein a subject with a P/O ratio of less than 0.9 is recommended for detoxification therapy.

9. The method of claim 1, wherein the subject is a human.

10. A method for identifying a chronic alcoholic comprising
determining a concentration of ethyl oleate (O) in a sample from a subject,
wherein a concentration of ethyl oleate (O) greater than 100 pmol/mL is indicative of a chronic alcoholic.

11. The method of claim 10, further comprising identifying a chronic alcoholic based on
determining a concentration of ethyl palmitate (P) in the sample, and
determining a P/O ratio of the concentration of ethyl palmitate to the concentration of ethyl oleate,
wherein a P/O ratio less than 0.9 is indicative of a chronic alcoholic.

12. The method of claim 10 or 11, further comprising identifying a chronic alcoholic based on
determining a concentration of total FAEE (T) in the sample, and
determining an O/T ratio of the concentration of ethyl oleate (O) to the concentration of total FAEE (T),
wherein an O/T ratio greater than 0.52 is indicative of a chronic alcoholic.

13. The method of claim 10, wherein the sample is blood.

14. The method of claim 10, wherein the sample is isolated from the subject within 4 days of the cessation of alcohol intake.

15. The method of claim 10, wherein the sample is isolated from the subject within 24 hours of the cessation of alcohol intake.

16. The method of claim 10, wherein the sample is isolated from the subject within 12 hours of the cessation of alcohol intake.

17. The method of claim 10, wherein a subject with a concentration of ethyl oleate greater than 100 pmol/mL is recommended for detoxification therapy.

18. The method of claim 10, wherein the subject is a human.

19. A method for identifying a chronic alcoholic comprising
determining a concentration of ethyl oleate (O) and a concentration of total FAEE (T) in a sample from a subject, and
determining an O/T ratio of the concentration of ethyl oleate (O) to the concentration of total FAEE (T) in the sample,
wherein an O/T ratio greater than 0.52 is indicative of a chronic alcoholic.

20. The method of claim 19, further comprising identifying a chronic alcoholic based on
determining a concentration of ethyl palmitate (P) in a sample from a subject, and
determining a P/O ratio of the concentration of ethyl palmitate (P) to the concentration of ethyl oleate (O) in the sample,
wherein a P/O ratio less than 0.9 is indicative of a chronic alcoholic.

21. The method of claim 19 or 20, further comprising identifying a chronic alcoholic based on
determining a concentration of ethyl oleate in a sample from a subject,
wherein a concentration of ethyl oleate greater than 100 pmol/mL is indicative of a chronic alcoholic.

22. The method of claim 19, wherein the sample is blood.

23. The method of claim 19, wherein the sample is isolated from the subject within 4 days of the cessation of alcohol intake.

24. The method of claim 19, wherein the sample is isolated from the subject within 24 hours of the cessation of alcohol intake.

25. The method of claim 19, wherein the sample is isolated from the subject within 12 hours of the cessation of alcohol intake.

26. The method of claim 19, wherein a subject with an O/T ratio of greater than 0.52 is recommended for detoxification therapy.

27. The method of claim 19, wherein the subject is a human.

28. A method for determining ethanol intake comprising
determining an amount of total FAEE in a liver sample from a subject
determining an amount of total FAEE in an adipose tissue sample from the subject, and
adding the amount of total FAEE in the liver sample to the amount of total FAEE in the adipose tissue sample to produce a combined total FAEE amount,
wherein a combined total FAEE amount of greater than 2000 pmol/g is indicative of ethanol intake by the subject.

29. The method of claim 28, wherein the subject is deceased.

30. The method of claim 28, wherein the subject is less than 2 years of age.

31. The method of claim 29, wherein the liver sample and the adipose tissue sample are harvested from the subject within 5 days of death.

32. The method of claim 29, wherein the liver sample and the adipose tissue sample are harvested from the subject within 3 days of death.

33. The method of claim 29, wherein the liver sample and the adipose tissue sample are harvested from the subject within 24 hours of death.

34. The method of claim 28, wherein the subject is a human.

35. A method for determining ethanol intake in a subject, comprising
determining an amount of total FAEE in a liver sample from the subject
determining an amount of total FAEE in an adipose tissue sample from the subject, and
determining the ratio of the amount of total liver FAEE to the amount of total adipose FAEE,
wherein a ratio of the amount of total liver FAEE to the amount of total adipose FAEE of at least 2 is indicative of ethanol intake by the subject.

36. The method of claim 35, wherein the subject is deceased and the method is a method for determining pre-mortem ethanol intake by the deceased subject.

37. The method of claim 35, wherein the subject is less than 2 years of age.

38. The method of claim 36, wherein the liver sample and the adipose tissue sample are harvested from the subject within 5 days of death.

39. The method of claim 36, wherein the liver sample and the adipose tissue sample are harvested from the subject within 3 days of death.

40. The method of claim 36, wherein the liver sample and the adipose tissue sample are harvested from the subject within 24 hours of death.

41. The method of claim 35, wherein the subject is a human.

42. The method of claim 35, wherein the subject has ethanol in the blood.

43. The method of claim 35, wherein the ethanol in the blood may be generated by bacteria.

44. The method of claim 35, wherein the amount of total liver FAEE is at least 10,000 pmol/gram.

45. A method for determining ethanol intake in a subject, comprising
determining an amount of ethyl arachidonate in a tissue selected from the group consisting of liver tissue and adipose tissue,
wherein an amount of ethyl arachidonate of at least 200 pmol/gram in the tissue is indicative of ethanol intake.

46. The method of claim 45, wherein the subject is deceased and the method is a method for determining pre-mortem ethanol intake by the deceased subject.

47. The method of claim 45, wherein the subject is less than 2 years of age.

48. The method of claim 46, wherein the tissue is harvested from the subject within 5 days of death.

49. The method of claim 46, wherein the tissue is harvested from the subject within 3 days of death.

50. The method of claim 46, wherein the tissue is harvested from the subject within 24 hours of death.

51. The method of claim 45, wherein the subject is a human.

52. The method of claim 45, wherein the subject has ethanol in the blood.

53. The method of claim 45, wherein the ethanol in the blood may be generated by bacteria.

54. The method of claim 45, wherein the amount of total liver FAEE is at least 10,000 pmol/gram.

55. The method of claim 45, further comprising determining a combined amount of total liver FAEE and total adipose FAEE, wherein a combined amount of at least 2000 pmol/gram or a ratio of the amount of total liver FAEE to the amount of total adipose FAEE of at least 2 is indicative of pre-mortem ethanol intake.

56. A kit for determining an amount of FAEE in a sample comprising
a collection tube,
a control amount of isolated FAEE,
an ice pack,
a thermal container, and
instructions for processing of the sample.

57. The kit of claim 56, wherein the FAEE is selected from the group consisting of ethyl oleate, ethyl palmitate, ethyl arachidonate, and total FAEE.

58. A method for identifying a binge drinker comprising
determining a concentration of ethyl oleate (O) and a concentration of ethyl palmitate (P) in a sample from a subject, and
determining a P/O ratio of the concentration of ethyl palmitate (P) to the concentration of ethyl oleate (O) in the sample,
wherein a P/O ratio greater than 1.0 is indicative of a binge drinker.

59. The method of claim 56, further comprising identifying a binge drinker based on
determining a concentration of ethyl oleate in a sample from the subject,
wherein a concentration of ethyl oleate less than 100 pmol/mL is indicative of a binge drinker.

60. The method of claim 58 or 59, further comprising
determining a concentration of total FAEE (T) in the sample and
determining an O/T ratio of the concentration of ethyl oleate (O) to the concentration of total FAEE (T) in the sample,
wherein an O/T ratio less than 0.52 is indicative of a binge drinker.

61. The method of claim 58, wherein the sample is blood.

62. The method of claim 58, wherein the sample is isolated from the subject within 2 days of the cessation of alcohol intake.

63. The method of claim 58, wherein the sample is isolated from the subject within 24 hours of the cessation of alcohol intake.

64. The method of claim 58, wherein the sample is isolated from the subject within 12 hours of the cessation of alcohol intake.

65. The method of claim 58, wherein a subject with a P/O ratio of greater than 1.0 is provided immediate medical attention.

66. The method of claim 58, wherein a subject with a P/O ratio of greater than 1.0 is placed on hemodialysis.

67. The method of claim 58, wherein the subject is a human.

68. A method for identifying a binge drinker comprising
determining a concentration of ethyl oleate (O) in a sample from a subject,
wherein a concentration of ethyl oleate less than 100 pmol/mL is indicative of a binge drinker.

69. The method of claim 66, further comprising identifying a binge drinker based on
determining a concentration of ethyl palmitate (P) in the sample, and
determining a P/O ratio of the concentration of ethyl palmitate (P) to the concentration of ethyl oleate (O),
wherein a P/O ratio greater than 1.0 is indicative of a binge drinker.

70. The method of claim 68 or 69, further comprising identifying a binge drinker based on
determining a concentration of total FAEE in the sample, and
determining an O/T ratio of the concentration of ethyl oleate (O) to the concentration of total FAEE (T),
wherein an O/T ratio less than 0.52 is indicative of a binge drinker.

71. The method of claim 68, wherein the sample is blood.

72. The method of claim 68, wherein the sample is isolated from the subject within 2 days of the cessation of alcohol intake.

73. The method of claim 68, wherein the sample is isolated from the subject within 24 hours of the cessation of alcohol intake.

74. The method of claim 68, wherein the sample is isolated from the subject within 12 hours of the cessation of alcohol intake.

75. The method of claim 68, wherein a subject with a concentration of ethyl oleate less than 100 pmol/mL is provided immediate medical attention.

76. The method of claim 68, wherein a subject with a concentration of ethyl oleate less than 100 pmol/mL is placed on hemodialysis.

77. The method of claim 68, wherein the subject is a human.

78. A method for identifying a binge drinker comprising
determining a concentration of ethyl oleate (O) and a concentration of total FAEE (T) in a sample from a subject, and
determining an O/T ratio of the concentration of ethyl oleate (O) to the concentration of total FAEE (T) in the sample,
wherein an O/T ratio less than 0.52 is indicative of a binge drinker.

79. The method of claim 78, further comprising
determining a concentration of ethyl palmitate (P) in a sample from a subject, and
determining a P/O ratio of the concentration of ethyl palmitate (P) to the concentration of ethyl oleate (O) in the sample,
wherein a P/O ratio greater than 1.0 is indicative of a binge drinker.

80. The method of claim 78 or 79, further comprising identifying a binge drinker based on
determining a concentration of ethyl oleate in a sample from the subject,
wherein a concentration of ethyl oleate less than 100 pmol/mL in the sample is indicative of a binge drinker.

81. The method of claim 78, wherein the sample is blood.

82. The method of claim 78, wherein the sample is isolated from the subject within 2 days of the cessation of alcohol intake.

83. The method of claim 78, wherein the sample is isolated from the subject within 24 hours of the cessation of alcohol intake.

84. The method of claim 78, wherein the sample is isolated from the subject within 12 hours of the cessation of alcohol intake.

85. The method of claim 78, wherein a subject with an O/T ratio less than 0.52 is provided immediate medical attention.

86. The method of claim 78, wherein a subject with an O/T ratio less than 0.52 is placed on hemodialysis.

87. The method of claim 78, wherein the subject is a human.

88. A computer program product, comprising:
a computer-readable medium; and
computer-readable signals stored on the computer-readable medium that define instructions that, as a result of being executed by a computer, instruct the computer to perform a process of determining ethanol intake by a subject, the process comprising steps (or acts) of
determining whether combined total amount of liver and adipose FAEE is at least 2000 pmol/gram;
determining whether ratio of total liver FAEE to total adipose FAEE is at least two;
determining whether amount of liver or adipose ethyl arachidonate is at least 200 pmol/g;
wherein i) a combined total amount of liver and adipose FAEE of at least 2000 pmol/gram, or ii) a ratio of total liver FAEE to total adipose FAEE of at least two and an amount of total liver FAEE of at least 10,000 pmol/g, or iii) an amount of ethyl arachidonate level in liver or adipose of at least 200 pmol/g, are each indicative of ethanol intake by a subject.

89. The computer program product of claim 88, further comprising the step of determining whether blood ethanol concentration is at least 10 mg/dL.

90. The computer program product of claim 88, further comprising the step of determining whether urine or vitreous ethanol levels are positive for ethanol.

91. The computer program product of claim 88, further comprising the step of determining the combined total amount of liver and adipose FAEE from data entry of total amount of liver FAEE and data entry of total amount of adipose FAEE.

92. The computer program product of claim 88, further comprising the step of determining the ratio of total liver FAEE to total adipose FAEE from data entry of total amount of liver FAEE and data entry of total amount of adipose FAEE.

93. The computer program product of claim 88, further comprising the step of determining whether the subject was a chronic alcoholic.

94. The computer program product of claim 88, further comprising the step of determining whether the subject was a binge drinker.

95. A method of determining ethanol intake by a subject, comprising
determining whether combined total amount of liver and adipose FAEE is at least 2000 pmol/gram;
determining whether ratio of total liver FAEE to total adipose FAEE is at least two;
determining whether amount of liver or adipose ethyl arachidonate is at least 200 pmol/g;
wherein i) a combined total amount of liver and adipose FAEE of at least 2000 pmol/gram, or ii) a ratio of total liver FAEE to total adipose FAEE of at least two and an amount of total liver FAEE of at least 10,000 pmol/g, or iii) an amount of ethyl arachidonate level in liver or adipose of at least 200 pmol/g, are each indicative of ethanol intake by a subject,
wherein the method is implemented on a computer.
